(19) United States
(12) Patent Application Publication
(10) Pub. No.: US 2002/0119458 A1
(43) Pub. Date: Aug. 29, 2002

(54) Novel computation with nucleic acid molecules, computer and software for computing

(75) Inventors: Akira Suyama, Hachioji-shi (JP); Yasubumi Sakakibara, Tokyo (JP); Nobuhiko Morimoto, Hachioji-shi (JP)

Correspondence Address: Scully, Scott, Murphy & Presser, 400 Garden City Plaza, Garden City, NY, 11530-0299, US

(73) Assignee: OLYMPUS OPTICAL CO., LTD., TOKYO (JP)

(21) Appl. No.: 09/893,205

(22) Filed: Jun. 27, 2001

(30) Foreign Application Priority Data
Dec. 15, 2000 (JP) 2000-382449
Dec. 27, 2000 (JP) 2000-399415

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

The present invention is to provide an information processing method using an operational nucleic acid, which comprises (a) converting arbitrary information into a nucleic acid molecule, (b) hybridizing the nucleic acid molecule obtained in (a) to an operational nucleic acid designed so as to express a logical equation indicating a condition to be detected, and extending the nucleic acid molecule hybridized, and (c) detecting a binding profile of the nucleic acid molecule included in the nucleic acid molecule extended in (b), thereby evaluating whether a solution of the logical equation is true or false. The present invention further provides an apparatus and a program for performing the information processing method.

### CROSS-REFERENCE TO RELATED APPLICATIONS

The application is based upon and claims the benefit of priority from the prior Japanese Patent Applications No. 2000-382449, filed Dec. 15, 2000; and No. 2000-399415, filed Dec. 27, 2000, the entire contents of both of which are incorporated herein by reference.

### BACKGROUND OF THE INVENTION

1. Field of the Invention

The present invention relates to a novel computation method using a nucleic acid molecule, a computer, and software for computing.

2. Description of the Related Art

Computers using semiconductor silicon have been improved in performance since their entry, and greatly contributed to mankind by carrying out complicated calculations at a low cost. The computers using semiconductor silicon are usually classified into a Neumann type which employs binary digits, 0 and 1, to perform a calculation.

In the field of computer science, there is a well-known investigation subject called a “NP-complete problem” which includes a “traveling salesman problem” and predicting a three-dimensional structure of a protein. To solve such problems completely, the following two approaches have been hitherto employed. In one of the approaches, all possible solutions are fit to a problem and whether they solve the problem is checked out. In the other approach, an approximate solution is obtained to solve the problem. In the former approach, much time is required for calculation to obtain a solution. Actually, the calculation time increases exponentially in proportion to the scale (complexity) of the problem. The latter approach is originally proposed to perform such a calculation at a high speed. Several algorithms have been proposed to obtain the approximate solution. However, it may not be possible to obtain an exact solution by using these algorithms. If anything, there is a danger that the right solution may be overlooked.

When the former approach is employed, calculations must be performed for all possible solutions. To perform the calculations at a high speed by use of presently available technique, numerous computers may be arranged in parallel and simultaneously operated.

However, this method accompanies the following problems. If the number of computers increases, power consumption inevitably increases, and further, a larger space is required for installing a large number of computers. To arrange numerous computers in parallel, many technical problems may arise, including how to transmit data between computers, and how to connect the computers with each other, etc.

On the other hand, to solve the problem which has not yet been clarified by the Neumann type computer, a new computer paradigm called “DNA computing” was proposed by Adleman in 1994 (Science, 266, 1021-4). Adleman employed DNA molecules to solve a small-scale traveling salesman problem. More specifically, the DNA molecules were constructed so as to correspond to a travelling route, and the DNA corresponding to the solution was selected from the DNA molecules thus constructed. Guanieri has reported another method in which DNA molecules are used to perform an add operation (Science, 273, 220-3). As described above, investigation has been performed as to the possibility of using DNA molecules in calculation.

It has been known that the following advantages are obtained if DNA is used in a calculation. For example, 1 pmol (=10⁻¹² mol) of short DNA molecules, each consisting of several tens of nucleotides, can be easily dissolved in 100 μL of buffer solution. The number of the short DNA molecules contained in the buffer solution reaches about 6×10¹¹. Assuming that the large number of DNA molecules interact upon each other to form DNA molecules corresponding to solutions, an extremely larger number of values can be calculated in parallel as compared to a conventional computer to obtain a desired solution. The interaction of DNA molecules takes place even in a solution of less than 1 mL. Therefore, even if a heating/cooling is applied to the solution of less than 1 mL, the energy consumption is little. If such a DNA computer is applied to solve a big problem with multi-variables, the processing speed of the DNA computer may exceed that of the Neumann type computer. Unfortunately, up to present, a DNA computer of practical use, efficiently using DNA molecules in calculation has not yet been developed.

### BRIEF SUMMARY OF THE INVENTION

Under the circumstances mentioned above, an object of the present invention is to provide a computation performed by a molecular computer, which is capable of evaluating a logical equation at a higher speed than a conventional electronic computer by use of parallelism of molecular operation and which is applicable to a reaction and a molecular computation.

The object of the present invention can be performed by the means, which is a method of processing information by using an operational nucleic acid. This method includes

(a) converting arbitrary information into a nucleic acid molecule;

(b) hybridizing the nucleic acid molecule obtained in the (a) to an operational nucleic acid designed so as to express a logical equation indicating a condition to be detected, and extending the nucleic acid molecule hybridized; and

(c) detecting a binding profile of the nucleic acid molecule included in the nucleic acid extended in the (b), thereby evaluating whether a solution of the logical equation is true or false.

By this method, it is possible to evaluate the presence or absence of the nucleic acid molecule by use of a nucleic acid having a specific sequence. Based on the evaluation, the operation of a logical equation can be performed at a high speed compared to a conventional electronic computer.

If this method is employed, it is possible to determine a genotype and an expression profile.

Another object of the present invention is to provide a computer for performing the information processing using the operational nucleic acid. The object of the present invention can be solved by the means: a molecular computer comprising an electronic operation section and a molecular operation section, in which the operation section substantially controls the function of the molecular operation section.

The computer of the present invention is useful not only for gene analysis but also for high-speed super parallel calculations for solving a hard mathematical problem such as the NP complete problem.

The present inventors have focused upon an original idea that a gene analysis is equivalent as a calculation if a nucleic acid molecule is used as input data. More specifically, a gene on an expressed mRNA or a genome is first converted into coding nucleic acids, and then, the coding nucleic acids may be used in screening and calculations such as logical OR, logical AND, and negation. Based on the calculations, it is possible to obtain a genotype and the gene expression conditions in a specific disease. Furthermore, a computer using the idea that gene analysis is applied to computer programming, can be provided.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently embodiments of the invention, and together with the general description given above and the detailed description of the embodiments given below, serve to explain the principles of the invention.

FIG. 1 is a schematic view showing the states of molecules in the reaction step of detecting the gene;

FIG. 2 is a schematic view showing the states of molecules in the reaction system where a target is present;

FIG. 3 is a schematic view showing the states of molecules in the step of capturing a strand with streptoavidin-bonded magnetic beads;

FIG. 4 is a schematic view showing the states of molecules in the step of extracting DCNᵢ;

FIG. 5 is a schematic view showing amplification of a sequence complementary to DCNᵢ obtained in the extraction step;

FIG. 6 is a schematic view showing the states of molecules in the step of capturing the amplified product obtained by the amplification step shown in FIG. 5;

FIG. 7 is a schematic view showing the states of molecules in the step of dissociating the double strand into single strands by heat denaturation;

FIG. 8 is a schematic view showing behavior of a molecule in the step of representing the information that a certain gene is expressed and present, by a presence molecule;

FIG. 9 is a schematic view showing the reaction of a presence/absence representing oligonucleotide and the presence molecule, in an initial step of detecting an unexpressed gene and representing it by an absence molecule;

FIG. 10 is a schematic view showing the state of the presence/absence representing oligonucleotide for use in detecting the unexpressed gene;

FIG. 11 is a schematic view showing the state of the presence/absence representing oligonucleotide in an extraction step;

FIG. 12 is a schematic view showing the state of the presence/absence representing oligonucleotide and DCN_{K}* in the step of capturing DCNₖ* by streptoavidin-bonded magnetic beads and extracting a desired gene with hybridization;

FIG. 13 is a schematic view showing an operational nucleic acid (a nucleic acid for use in a computer operation);

FIG. 14 is a schematic view showing the state of the operational nucleic acid and DCN₃, in the hybridization step with a presence molecule, and an absence molecule;

FIG. 15 is a schematic view showing the state of the operational nucleic acid in the step of extending the presence molecule hybridized with the operational nucleic acid, after the step of FIG. 14;

FIG. 16 is a schematic view showing the states of molecules in the step of detecting calculation results by maker oligonucleotides M₁ and M₂;

FIG. 17 is a schematic view showing the states of molecules in the step of extracting and recovering the absence molecule for amplifying it;

FIG. 18 is a schematic view showing the states of molecules in a PCR amplification step for the absence molecule;

FIG. 19 is a schematic view showing the states of molecules in the step of capturing the amplified product produced in the step of FIG. 18;

FIG. 20 is a schematic view showing the states of molecules in the step of dissociating a single strand of the amplified product recovered in the step of FIG. 19;

FIG. 21 is a schematic view showing the states of molecules and the absence molecule in the step of hybridizing the absence molecule to the single strand obtained in FIG. 20:

FIG. 22 is a schematic view showing a complementary ligation nucleic acid and a part of the operational nucleic acid to be ligated for use in preparing a random library for the operational nucleic acid;

FIG. 23 is a flow chart showing an encode reaction and a decode reaction for gene analysis;

FIG. 24 is a chart showing a molecular design for gene analysis;

FIG. 25 is a block diagram showing a structure of a molecular computer of the present invention;

FIG. 26 is a flow chart showing the processing procedure of the molecular computer of the present invention;

FIG. 27 is a block diagram showing a structure of the molecular computer of the present invention;

FIG. 28 is a block diagram showing the arrangement of sections of the molecular computer of the present invention;

FIG. 29 is a schematic flow chart of command operation;

FIG. 30 is a schematic flow chart of command operation;

FIG. 31 is a schematic flow chart of command operation;

FIG. 32 is a schematic flow chart of command operation;

FIG. 33 is a flow chart showing a flow of a program;

FIG. 34 is a conceptual view showing a method of identifying a sequence; and

FIG. 35 is a chart showing the results of identified sequences.

### DETAILED DESCRIPTION OF THE INVENTION

In an aspect of the present invention, there is provided a data parallel computation method using nucleic acid molecules. More specifically, there is provided a method for processing data using the nucleic acid molecules as a medium carrying data. In this method, an operation of data carried by the nucleic acid molecules is performed by making use of a reaction such as an enzymatic reaction or a hybridization reaction of the nucleic acid molecules.

The “nucleic acid molecule” and the “molecule” used herein refer to DNA and RNA including cDNA, a genome DNA, synthetic DNA, mRNA, total RNA, hnRNA and synthetic RNA. The terms “nucleic acid molecule” and “molecule” can be interchangeably used.

The parallelism of information processing performed by the nucleic acid molecules is extremely high. For example, 1 mL of 100 μM DNA oligonucleotide solution, which is frequently used in the molecular biology, contains 6×10¹⁶ DNA oligonucleotide molecules. Provided that a single DNA oligonucleotide molecule represent one bit (letter), a storage capacity of 60,000,000 G bite can be provided. In this case, it is assumed that execution of a single command takes 10³ seconds. If the same command is simultaneously executed by 6×10⁶ DNA molecules, 6×10¹³ commands come to be executed per second. As exemplified, the parallelism of the information processing performed by nucleic acid molecules is extremely high. To explain more specifically, if data and programs are expressed by use of nucleic acid molecules and commands are executed by molecular reactions of the nucleic acid molecules, the storage capacity and parallelism of information processing thus attained become extraordinarily larger than those of conventional electronic computers.

According to another aspect of the present invention, nucleic acid analysis is disclosed. In this analysis, the nucleic acid can be analyzed base on a computation using such nucleic acid molecules.

According to still another aspect of the present invention, a general methodology for genomic information analysis based on the computation using nucleic acids. In particular, the genomic information analysis has the following advantages. First, a desired molecule having a nucleotide sequence arbitrarily designed is assigned to the nucleotide sequence of a specific gene. In accordance with the aforementioned assignment manner, specific nucleic acid sequences are represented by the arbitrarily designed sequences. Since the designed sequences have substantially an equal heat stability, they can be efficiently used in operation. If this method is employed, the reaction conditions can be set with a high degree of freedom, and the reaction can be accurately performed.

In a further aspect of the invention, a computer for carrying out the aforementioned method is disclosed. In the information processing method and analysis method, the reaction can be performed by manually manipulating nucleic acid molecules and various reagents. However, these manipulations may be automatically performed by an apparatus except input operations. By using such automatic operation apparatus, information processing, computation, and gene or genomic analysis can be performed. Such an apparatus is also one of the aspects of the present invention. The apparatus is applicable to general molecular calculations.

Now, the present invention will be more specifically explained below.

I. Calculation method

1. First embodiment

(1) General Outline

A first embodiment of the present invention will be explained. The first embodiment shows a gene analysis for determining the presence or absence of a gene by performing an operation using nucleic acids.

The analysis will be outlined below. First, a cDNA group is prepared based on a gene group expressed in a cell. Information regarding an expressed gene contained in the cDNA group thus prepared and an unexpressed gene not contained in it, more specifically, information on the presence/absence of a target gene is converted into another expression form such as a DNA molecule having an artificially designed sequence. The DNA molecule obtained by data conversion is hybridized to an operation nucleic acid. The operation analysis is performed in accordance with the aforementioned process. The DNA molecule used herein acts as a kind of signal which represents the presence/absence of a specific gene of interest. For example, if the presence of the target gene is confirmed, it can be determined that the target gene is actually expressed. Conversely, if the absence of the target gene is confirmed, it can be determined that the target gene is not expressed. Accordingly, in this analysis method, it is possible not only to detect the target molecule contained in a sample but also to obtain the information that the target gene is not present in a sample.

(1.1) Preparation

In the computation method according to the first aspect of the present invention, the following molecules must be prepared before a computation is virtually performed. The molecules are prepared by a known method.

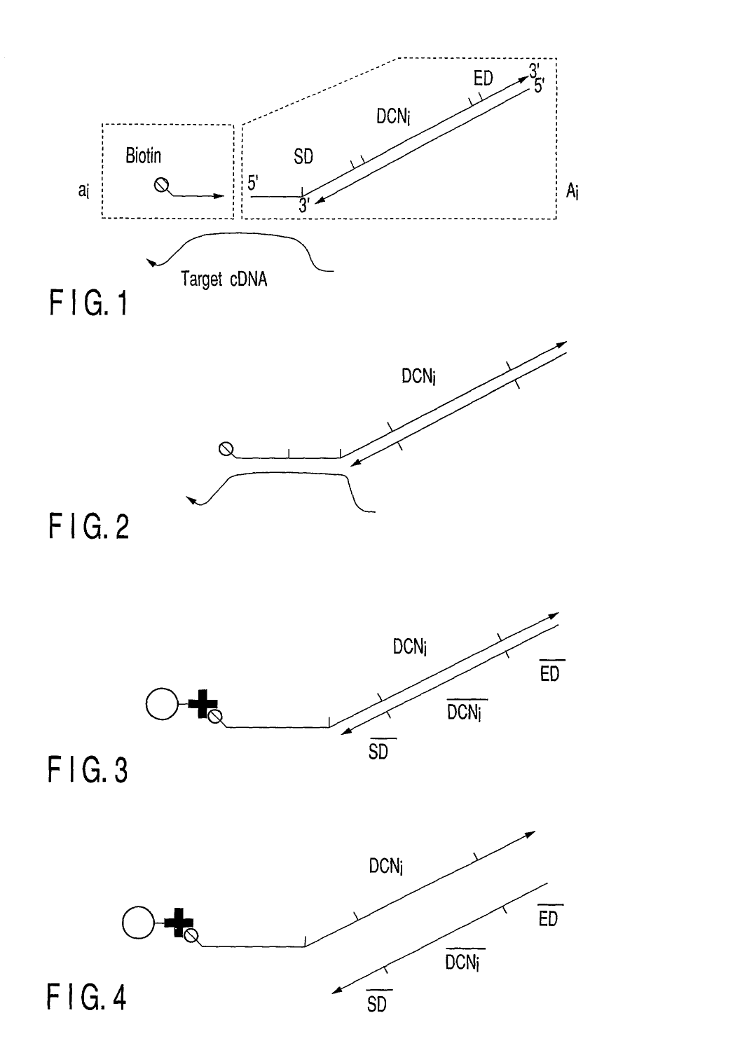

First, two probes, namely, aᵢ and Aᵢ, (surrounded by a broken line in FIG. 1) are prepared for detecting a cDNA molecule contained in a solution. Probe aᵢ is an oligonucleotide containing a sequence complementary to a part of the sequence of a target cDNA. The 5′ end of Probe aᵢ is labeled with biotin. Probe Aᵢ is an oligonucleotide partially having a double-stranded region due to hybridization. Of the double strands, one has artificially designed sequences, SD, DCNᵢ and ED, at a side near the 3′ end, and a sequence which is complementary to a part of the sequence of the target cDNA at the 5′ end. It is desirable that the nucleotides complementary to the 5′ end of the sequence A₁ and the nucleotides complementary to the 3′ end of the sequence a₁ should be arranged on the target cDNA next to each other. Furthermore, the nucleic sequences artificially designed (SD, DCNᵢ and ED) are arranged at a site closer to the 3′ end than the aforementioned complementary sequence. The 5′ end of the strand of Probe A₁ having the complementary sequence to the target cDNA, is phosphorylated. The other strand constituting the double strand Aᵢ region is an oligonucleotide having complementary sequences to the SD, DCNᵢ and ED. The probes aᵢ and Aᵢ are arbitrarily designed with respect to every target gene to be detected. The term “target gene” used herein is a gene whose presence/absence is to be detected in a solution. The sequence DCNᵢ is designed such that it varies depending upon a target. In this case, SD and ED sequences are common in all probes Aᵢ. These artificial sequences can be arbitrarily designed. This means that the Tm value of these sequences can be set at a desired value. Since the heat stability of the sequences can be set at the same in this manner, a hybridization reaction can be performed with little occurrence of mishybridization.

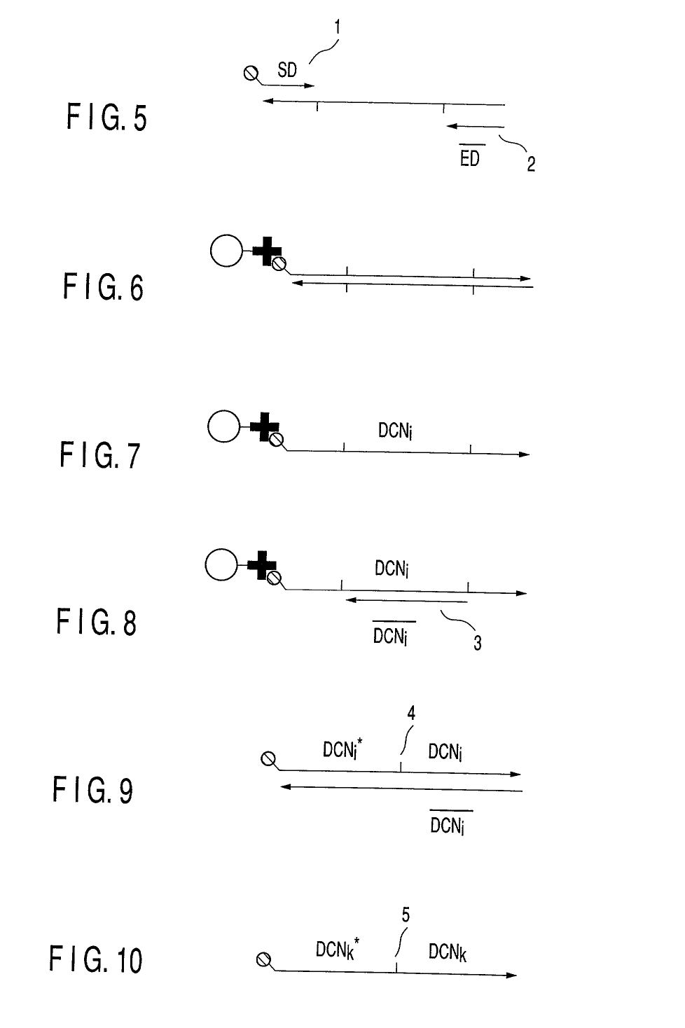

Furthermore, primers 1, 2 (shown in FIG. 5) are required. The primer 1 has the same sequence as the SD sequence whose 5′ end is labeled with biotin. The primer 2 has a complementary sequence to the ED sequence (see FIG. 5).

Moreover, an oligonucleotide 3 (FIG. 8) and an oligonucleotide 6 (FIG. 12) are required. The oligonucleotide 3 (hereinafter referred to as a “presence oligonucleotide”) has a complementary sequence to DCNᵢ indicating “a target is present”. The oligonucleotide 6 (hereinafter, referred to as an “absence oligonucleotide”) has a complementary sequence to DCNᵢ* indicating “a target is absent”.

In addition, a presence/absence representing oligonucleotide 4 (shown in FIG. 9) is required. The oligonucleotide 4 has DCNᵢ* and DCNᵢ sequences in the order from the 5′ end. The DCNᵢ* sequence is a nucleotide sequence artificially designed so as to correspond to DCNᵢ based on a predetermined rule and different in sequence from the DCNᵢ.

(1.2) Conversion to the presence molecule and absence molecule

In the method of the present invention, the information “a specific target molecule is present in a sample” is represented by the “presence molecule”, whereas the information “the specific target molecule is not present in the sample” is represented by the “absence molecule”. The term “presence molecule” used herein and the term of “presence oligonucleotide” can be interchangeably used. Similarly, the terms “absence molecule” and “absence oligonucleotide” can be interchangeably used.

Now, the information of the presence/absence of a molecule is converted into the presence or absence molecule in accordance with the method shown in FIGS. 1 to 12. FIGS. 1 to 23 schematically show molecules present in a system of each step.

In the figures, DNA is indicated by an arrow. The proximal end of the arrow is the 5′ end of DNA and the distal end of the arrow is the 3′ end. The nucleotide sequence is partitioned by a short perpendicular line(s). Names of sequences are represented by alphabets such as “a”, “A”, and “DCN” arranged near the arrow in the figures. Alphabets such as “i” and “k” additionally attached to alphabets “a”, “A”, and “DCN” are integers. An arbitrary sequence is indicated by the alphabets “i” or “k” and the correspondence between sequences are shown by the integer of “i” or “k”. For the sake of convenience, alphabet “i” indicates an expressed gene and alphabet “k” indicates an unexpressed gene. On the other hand, the line drawn above the name of a sequence indicates a complementary sequence. A hatched circle indicates a biotin molecule. A large open circle indicates magnetic beads. A solid cross placed at a right-hand side of the magnetic beads schematically indicates a streptoavidin molecule capable of specifically binding to the biotin molecule fixed on the magnetic beads.

a. Conversion of the information “target is present” into the “presence molecule”

When the tart is present, the information “target is present” is converted into the “presence molecule” by sequential steps of FIGS. 1 to 19.

Referring now to FIG. 1, the probes aᵢ and Aᵢ (synthesized as mentioned above) are reacted with a target cDNA in a reaction buffer containing an enzyme, such as Taq ligase, maintaining a high activity at a high temperature. The ligation reaction is performed at a temperature at which a double-stranded region of the Aᵢ oligonucleotide is not dissociated. When the target cDNA is present, the probes aᵢ and Aᵢ are ligated by the action of Taq ligase as shown in FIG. 2 after the reaction. Next, the ligated aᵢ-Aᵢ oligonucleotide is extracted by the magnetic beads having streptoavidin bonded on the surface, from the reaction solution, as shown in FIG. 3. At that time, an unreacted aᵢ molecule is captured by the beads but will not be participated in a later reaction.

Subsequently, a complementary strand of the Aᵢ portion is extracted by isolating it from the aᵢ-Aᵢ ligation molecule (captured by the magnetic beads) with heat application (FIG. 4). If cDNA is present in an initial solution, it is possible to extract the oligonucleotide containing a complementary sequence to a DCNᵢ sequence corresponding to the cDNA, by the aforementioned operation (FIG. 4). Using the extracted oligonucleotide as a template, a PCR amplification reaction is performed as shown in FIG. 5 by use of two primers. One of the two primers has an identical sequence to SD and labeled with biotin at the 5′ end. The other has an identical sequence to ED (FIG. 5). As a result, the DCNᵢ sequence (which proves the presence of a target gene) is amplified.

The double-stranded PCR product is captured by streptoavidin-fixed magnetic beads, as shown in FIG. 6 (FIG. 6). Heat is applied to the double-stranded PCR product thus captured, thereby dissociating it into single strands. The complementary strand thus dissociated is removed by exchanging a buffer solution (FIG. 7). Subsequently, as shown in FIG. 8, the presence oligonucleotide having a complementary sequence to DCNᵢ is hybridized with the PCR product captured by the beads (FIG. 8). After the hybridization, presence oligonucleotides excessively present are removed. Subsequently, heat is applied again to the hybridized PCR product. As a result, the complementary strand (namely, presence oligonucleotide 3) to DCNᵢ is extracted into a buffer from the strand captured by the beads.

The presence oligonucleotide 3 having a complementary sequence to the DCNᵢ extracted herein is the “presence molecule” indicating the presence of a target gene in the original cDNA solution.

b. Conversion of the information “a target is absent” into the “absence molecule”

In the step a, the information “the target gene is present” is converted into the presence molecule (presence oligonucleotide) indicating that the target gene is present. Thereafter, the information “the target gene is absent” is converted into the absence molecule (absence oligonucleotide). When the target gene is not present, the absence oligonucleotide is extracted. The extraction is performed as follows:

First, a presence/absence representing oligonucleotide 4 (as shown in FIG. 9) is prepared with respect to DCN of the target gene. As mentioned above, the presence/absence representing oligonucleotide 4 is artificially prepared so as to correspond to DCNᵢ. The presence/absence oligonucleotide also has a nucleotide sequence, DCNᵢ* at the 5′ end side and DCNᵢ at the 3′ end side of DCNᵢ*. The sequence DCNᵢ* is artificially designed so as to correspond to DCNᵢ based on a predetermined rule and differs in sequence from DCNᵢ. When the presence/absence representing oligonucleotide is subjected to a hybridization reaction with the “presence molecule”, the information “a target is absent” can be converted into a detectable “absence molecule”.

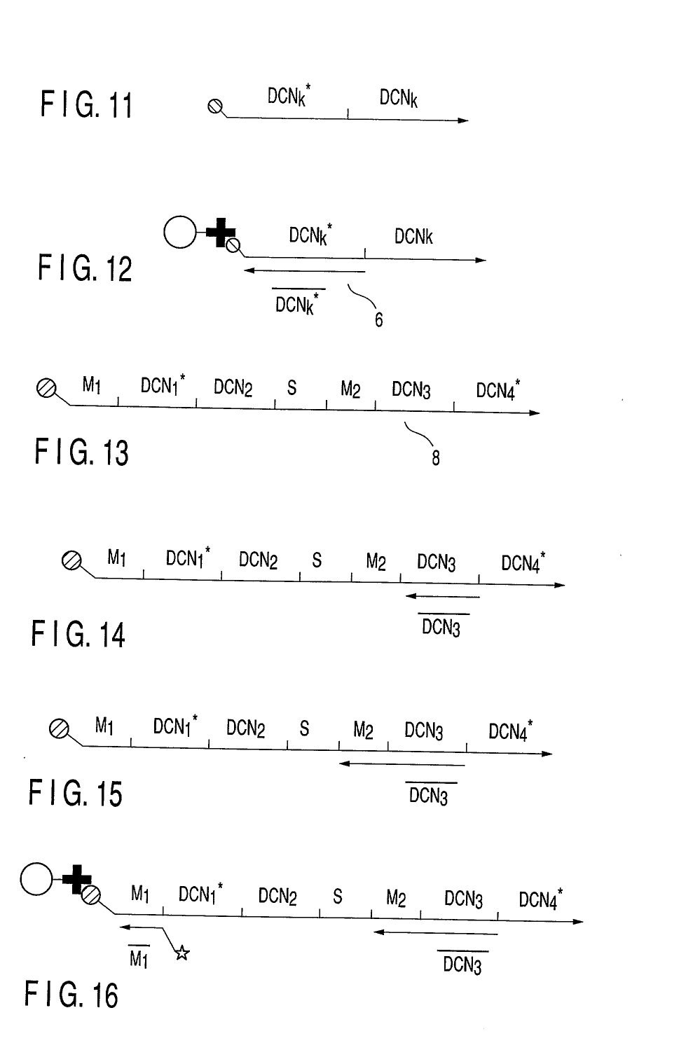

The hybridization reaction is performed as shown in FIG. 9. First, the presence oligonucleotide 3 (DCNᵢ) which corresponds to an expressed gene and extracted in the step of FIG. 8, is hybridized to the presence/absence representing oligonucleotide 4. Thereafter, the hybridized DCN_{I} is extended with polymerase (FIG. 9). As a result, DCNᵢ (corresponding to the expressed gene) extends up to the end of the sequence of DCNᵢ* to synthesize a complementary strand (FIG. 9). In contrast, when the target molecule is not expressed (indicated by DCNₖ herein), as shown in FIG. 10, the oligonucleotide complementary to DCNₖ is not present in a reaction solution. As a result, the presence/absence representing oligonucleotide 5 is present in a single-stranded form (FIG. 10). A mixture containing the double-stranded presence/absence representing oligonucleotide and the single-stranded presence/absence representing oligonucleotide is then loaded onto a column containing hydroxyapatite. As a result, the single-stranded presence/absence representing oligonucleotide 5 alone is extracted (FIG. 11).

The extracted presence/absence representing oligonucleotide 5 having DCNₖ (corresponding to the unexpressed gene) is captured by streptoavidin-bonded magnetic beads (FIG. 12). Next, in the same manner as in the case where the presence oligonucleotide 3 alone is extracted, an oligonucleotide 6 complementary to DCNₖ* is hybridized. After oligonucleotides 6 excessively present are removed, only absence oligonucleotides 6 hybridized with DCNₖ* (indicating that the gene is not unexpressed) can be extracted (FIG. 12).

The step of obtaining the absence oligonucleotide 6 can be also carried out as described below.

First, a fluorescent molecule such as FITC is labeled at the 5′ end of the DCNᵢ oligonucleotide. Thereafter, a hybridization reaction is performed on a DNA microarray containing a probe having a sequence complementary to DCNᵢ. The fluorescent molecule of the hybridization reaction is read out by a scanner. In this way, which DCNᵢ is present can be detected. At the same time, DCNₖ (indicating that a target is absent) can be detected. Based on these data, the absence oligonucleotide 6 (DCNₖ*) is prepared which will be subjected to the following operation. From the aforementioned steps, the information that “a nucleic acid (target) is absent” can be converted into the “absence nucleic acid” (representing the absence of a target), which is designed so as to correspond to the target nucleic acid in accordance with a predetermined rule. From the aforementioned procedure, logic operation can be performed by using an operational nucleic acids.

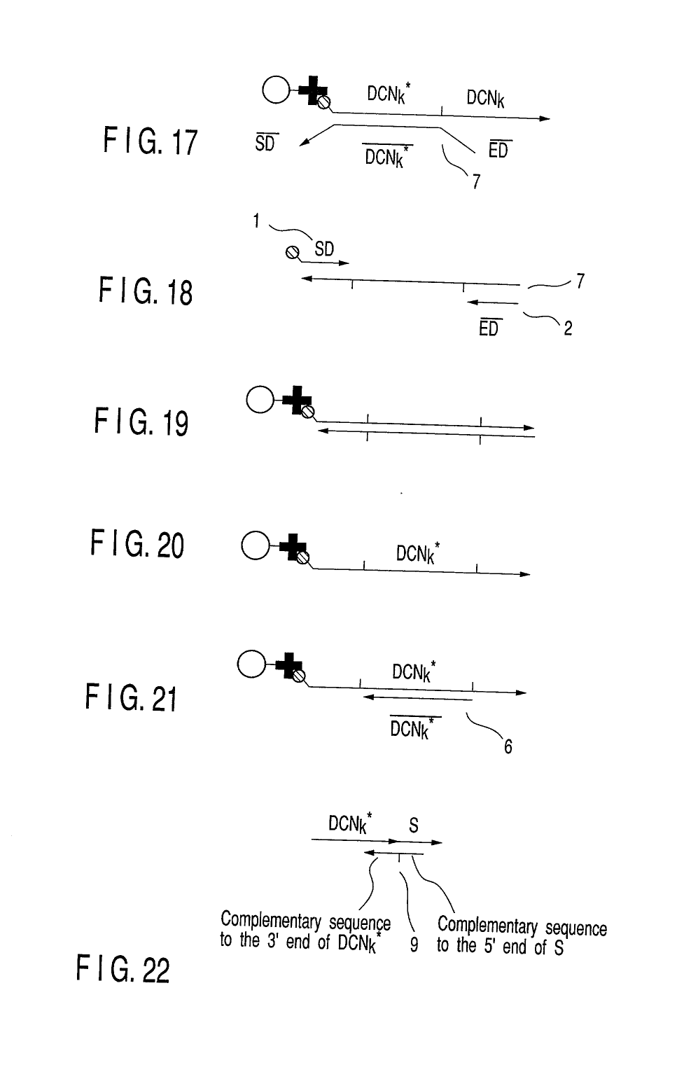

In the step of preparing the absence oligonucleotide 6, the absence oligonucleotide 6 may be amplified by using a single stranded presence/absence representing oligonucleotide 5 as a template. The amplification may be performed in accordance with the steps shown in FIGS. 17-22. In the figures, a molecule(s) is schematically shown. The detailed explanations of reference symbols within the figures are the same as described above. First, a single-stranded presence/absence representing oligonucleotide 5 is extracted in accordance with the step of FIG. 11 (FIG. 11). The presence/absence representing oligonucleotide 5 is hybridized with an oligonucleotide 7. The oligonucleotide 7 has a sequence complementary to DCNₖ* which is bonded to a sequence complementary to the SD sequence at the 3′ end and a sequence complementary to the ED sequence at the 5′ end. As a result, the absence oligonucleotide 6 is extracted. Subsequently, DCNₖ* is amplified by the steps of FIGS. 18 to 22, in the same manner as in the case of amplifying the presence oligonucleotide 3. More specifically, in the step of FIG. 18, the oligonucleotide 7 (obtained in the step of FIG. 17) is amplified by primers 1 and 2. The primer 1 has the SD sequence labeled with biotin. The primer 2 has a sequence complementary to the ED sequence (FIG. 18). Thereafter, the resultant PCR product is recovered by bonding biotin to a streptoavidin molecule (FIG. 19). Subsequently, heat denaturation is performed to convert the PCR product into a single-stranded PCR product (FIG. 20). Subsequently, the absence oligonucleotide having a sequence complementary to DCNₖ* is hybridized to the PCR product captured by beads (FIG. 21). After the hybridization, the absence oligonucleotides excessively present are removed. Heat is applied again to extract a complementary strand to DCNₖ* (absence oligonucleotide 6) captured by beads into a buffer. The absence oligonucleotide 6 extracted represents that a target gene is not present in an original cDNA solution.

(1.3) Operation step

In the operation step, the presence molecule and the absence molecule obtained above are hybridized to the operational nucleic acid, and then, a complementary strand of the hybridized molecule is synthesized. According to this method, parallel computation is performed by solving an operation expression expressing desired conditions to thereby obtain a solution satisfying the operational conditions.

As an example, Equation 1 is used as an operation expression. Specific sequences, DCN₁, DCN₂, DCN₃ and DCN₄ are target sequences. Equation 1 is a logical equation indicating the conditions as to the presence and absence of the target sequences. Equation 1 is operated based on the hybridization reaction of the operational nucleic acid with the presence molecule and the absence molecule and extension of the hybridized molecule. The obtained values are evaluated. Equation 1 expresses desired conditions as to the presence and absence of DCN₁, DCN₂, DCN₃ and DCN₄. In brief, solving Equation 1 of the present invention means that the presence and absence of the target sequences in a sample are identified simultaneously.

Equation 1

(DCN₁ [symbol] [symbol] DCN₂) [symbol] ( [symbol] DCN₃ [symbol] DCN₄)

where “ [symbol] ” denotes “negation”, “ [symbol] ” denotes “AND”, “ [symbol] ” denotes “OR”.

When the predetermined conditions for the operational nucleic acid are satisfied, the value of Equation 1 becomes “1” that is, “true”. Alternatively, when the conditions are not satisfied, the value of Equation 1 becomes “0”, that is “false”.

FIG. 13 shows a sequence structure of an operational nucleic acid 8. The operational nucleic acid 8 is a single-stranded oligonucleotide (shown by an arrow in the figure). The arrow proceeds from the 5′ end to the 3′ end. A biotin molecule is attached to the 5′ end. The operational nucleic acid 8 includes a plurality of units. The units (nucleotide sequences) are M₁, DCN₁*, DCN2, S, M₂, DCN₃, and DCN₄*, which are arranged in this order from the 5′ end. M1 is a sequence to which a marker molecule is to be linked. DCN₁* is a sequence for detecting the oligonucleotide which is obtained when DCN₁ is absent. S is a stopper sequence which terminates extension of a complementary strand by polymerase. M₂ is a sequence to which a second marker is to be attached. The arrangement order of the sequence units of the operational nucleic acid almost corresponds to that of the items of the logical equation. “Negation” of the logical equation is also expressed by the sequence itself. To explain more specifically, in the case where the presence of the “DCN₄” sequence is denied, the sequence “DCN₄*” is used. The sequence “DCN₄*” used herein is artificially designed so as to correspond to the sequence “DCN₄” mentioned above. Logical “OR” is expressed by the sequence S. However, a specific sequence expressing logical “AND” is not used on the operational nucleic acid. When the conditions are satisfied as shown in Table 1, value 1 is obtained as calculation results of the operational nucleic acid. In the table 1, a symbol “-” denotes that either “expressed” or “unexpressed” is acceptable.

TABLE 1

| DCN₁ | DCN₂ | DCN₃ | DCN₄ | Value |
|---|---|---|---|---|
| expressed | unexpressed | — | — | 1 |
| — | — | unexpressed | expressed | 1 |

Now, the logical operation is performed by carrying out nucleic acid reactions. The steps of the nucleic acid reaction shown in FIG. 14 to FIG. 16 will be explained. The operation reaction is performed in accordance with the following procedure. Operation is performed in accordance with Equation 1, an operational nucleic acid having the sequence units (shown in FIG. 13) is prepared. A single tube contains a single type of operational nucleic acid. To the tube, a solution containing both the presence oligonucleotide and the absence oligonucleotide is added, and a hybridization reaction is performed (FIG. 14). Assuming that DCN₁, DCN₃ and DCN₄ are present and DCN₂ is absent, the operational nucleic acid is hybridized with DCN₃ alone (FIG. 14). Furthermore, after the hybridization reaction, an extension reaction is performed with an enzyme, Taq polymerase, having an activity even at a high temperature, under the conditions causing no mishybridization. As a result, the extension reaction proceeds up to M₂ sequence unit and stops at the S sequence. The region from DCN₃ to M₂ is double-stranded (FIG. 15).

Next, after completion of the reaction, the operational nucleic acid is captured by the streptoavidin magnetic beads (FIG. 16). Finally, a marker oligonucleotide is hybridized to the operational nucleic acid as a detection reaction (FIG. 16). FIG. 16 shows the operational nucleic acid fixed on a carrier beads. A biotin molecule at the 5′ end of the operational nucleic acid is linked to a streptoavidin molecule on the carrier beads. Furthermore, marker oligonucleotides M₁ and M₂ are prepared which are complementary to marker detection sequences. A fluorescent molecule is attached to the 5′ end of each of these marker oligonucleotides M₁ and M₂. In this case, the M₁ sequence of the operational nucleic acid is not double stranded, the marker oligonucleotide can be hydridized to the operational nucleic acid (FIG. 16). After unbound marker oligonucleotides are removed, the fluorescence of the beads is checked. Since the marker oligonucleotide M₁ is hybridized and emits fluorescence in this case, the result of the operation, that is, value of the logical equation, becomes “1”.

In the aforementioned example, the S sequence is used as a stopper. However, the S sequence is not necessarily arranged. In place of the S sequence, an artificially-designed nucleotide may be used. The artificially-designed nucleotide has a base whose complementary strand cannot be formed. In this case, S sequence or the artificially-designed nucleotide is flanked with other units of the operational nucleic acid. For example, the artificially-designed stopper nucleotide is designed such that a cytosine base is included in it but not included in the other sequence units. In this case, the extension reaction (performed later, as shown in FIG. 15) on an operational nucleic acid stops at the artificially designed nucleotide if a dGTP (described later) is not added as a building-block monomer in the extension reaction performed thereafter. Alternatively, the stopper sequence may be constituted of a guanine-cytosine continuous bases. This is because a polymerase extension reaction tends to stop at the guanine-cytosine continuous bases. Moreover, PNA complementary to the S sequence may be previously hybridized to the operational nucleic acid. The DNA/PNA hybrid is more stable than a DNA/DNA hybrid. Therefore, if a polymerase having a 5′ exonuclease activity is used, the PNA cannot be removed. Therefore, the S sequence formed of the DNA/PNA hybrid works as a stopper.

Furthermore, many types of fluorescent pigments may be attached to the marker oligonucleotide. Up to present, since many fluorescent pigments have been developed, difference nucleic acids may be labeled with different fluorescent pigments, respectively. If this method is employed, a large number of operational nucleic acids can be distinguishably labeled at the same time. For example, if M₁ maker oligonucleotide and the M₂ marker oligonucleotide are labeled with different fluorescent pigments in the embodiment, it is possible to know which condition within parenthesis of the logical equation is satisfied based on which fluorescent pigment is detected. Moreover, different marker sequences are used depending upon operational nucleic acids and marker oligonucleotides are labeled with different fluorescent molecules, different operation reactions of a plurality of types of operational nucleic acids can be simultaneously performed in a single tube. On the other hand, the intensity of fluorescence increases in proportional to how sufficiently the logical equation (expressed by an operational nucleic acid) is satisfied. Hence, it is possible to know the satisfactory level of the logical equation.

To obtain the operation results, the following technique may be employed. In the step of amplifying the presence oligonucleotide and absence oligonucleotide, if a PCR reaction is performed in sufficient number of cycles until the amplification reaches saturation, binary logical operation can be made by using two values: “1” representing “presence” and “0” representing “absence”. On the other hand, if the amplification is performed so as not to reach the saturation, that is, if the number of cycles for the PCR reaction is limited so as to obtain the PCR product in an amount proportional to an original amount of cDNA, the logical equation can be evaluated as to the probability in the zone of [0, 1]. For example, in the case of an expressed gene, the results are obtained in proportional to the expression amount of the expressed gene. In the case of a genomic sequence, it is possible to know the probability whether the genomic sequence is a hetero zygote or a homozygote, based on the operational results.

Furthermore, operational nucleic acids may be fixed on a substrate in the form of micro spots like a DNA microarray. The addressing of the micro spots may be made such that the positions of the microspots correspond to the logical equation(s) of the operational nucleic acids. In this case, the marker oligonucleotide is preferably tagged with a fluorescent label. If the operation reaction mentioned above is performed by using operational nucleic acids fixed on the microarray, the operation results can be read out by a readout scanner of a DNA microarray.

The marker oligonucleotide may be labeled with biotin. In this case, biotin is not attached to the 5′ end of the operational nucleic acid. Instead, the operational nucleic acid may include a restriction enzyme recognition sequence for cloning at the 3′ end and the 5′ end. In addition, the step of capturing the operational nucleic acid by streptoavidin-magnetic beads (shown in FIG. 16) is not performed in the reaction. The detection herein is performed by hybridizing a marker oligonucleotide to the operational nucleic acid, capturing both marker oligonucleotides hybridized and nonhybridized to the operational nucleic acid by streptoavidin-magnetic beads, cloning the captured operational nucleic acids, and reading out the sequences by a sequencer. By this step, the operational nucleic acid(s) giving the operation result “1” is identified. If the operation is made in this manner, the reactions of a plurality of types of operational nucleic acids can be performed in a single tube.

In this embodiment, four target sequences are used. However, more than 4 target sequences may be used. Furthermore, biotin and streptoavidin are used herein as tags for use in recovering. However, the tags are not limited to them. Any substances may be used as a tag as long as they exhibit a high affinity with each other.

2. Second embodiment

According to a preferable embodiment of the present invention, an orthonormal sequence may be used as a DCN sequence in the method of First embodiment. The term “orthonormal sequence” is a nucleotide sequence artificially designed. The term “normal” means that sequences have the same melting temperature (Tₘ). The term “ortho” means that no mishybridization occurs and that stable structure is not formed within a molecule.

For example, to obtain the orthonormal sequence of 15 nucleotides, first, sequences each consisting of arbitrary-chosen 5 nucleotides are prepared. The short sequence of 5 nucleotides is called “tuple”. The types of tuples are 4⁵=1024. From the 1024 types of tuples, three tuples are selected and connected to each other to form a 15 nucleotide sequence. The tuples complementary to these three tuples employed herein will not used thereafter. When a set of 15 nucleotides is prepared, care must be taken to set the melting temperature of the 15 nucleotide sequence within ±3° C. In addition, it must be check on the possibility that the 15 nucleotide sequence will form a stable structure within the molecule. If it takes the stable form, such 15 nucleotide sequence must be eliminated.

Finally, all sequences of 15 nucleotides are checked as to the possibility that they will pair up with each other to form a double strand. The 15 nucleotide sequences thus prepared independently perform hybridization reactions at an appropriate temperature without forming a hybrid between them, even if they are used in a mixture. Hence, if nucleic acids aᵢ and Aᵢ are constructed such that the orthonormal sequence corresponds to the nucleotide sequence of a specific gene based on a specific rule and subjected to the reaction to be performed in accordance with the first embodiment, the reaction conditions can be further simplified and thereby an operation reaction can be more accurately performed.

3. Third embodiment

According to a preferable embodiment of the present invention, the method of the present invention can be used to check a genotype which is determined based on which nucleotide sequences are present at which loci. In this embodiment, the method of determining a genotype will be described.

First, a logical equation is set so as to correspond to the genotype. Based on the logical equation, an operational nucleic acid is designed. If the operational nucleic acid is used, it is possible to determine the genotype without using an electronic calculator and an evaluation table. For example, assuming that a gene having base A at locus 1, base T at locus 2, and base G at locus 3 can be determined as genotype A; a gene having base A at locus 1, base C at locus 2, and base T at locus 3 can be determined as genotype B; and a gene having base A at locus 1, base C at locus 2, base G at locus 3 can be determined as genotype C, logical equations satisfying the aforementioned three cases are shown in Table 2, the right end column.

TABLE 2

| | | | | Logical |
|---|---|---|---|---|
| | Locus 1 | Locus 2 | Locus 3 | Equation |
| Genotype A | A | T | G | A [symbol] T [symbol] G |
| Genotype B | A | C | T | A [symbol] C [symbol] T |
| Genotype C | A | C | G | A [symbol] C [symbol] G |

Each of the genotypes is determined by performing an operation reaction using the operational nucleic acid corresponding to each logical equation. First, when a specific sequence is present at a locus, the value of a term of the logical equation is set at “1”, and when it is absent, the value is set at “0”. In this case, if the total value of the equation results in 1, the genotype represented by the equation is the evaluation result. If the method of the present invention using nucleic acids in operation, it is possible to determine a genotype without using a complicated table and an electronic computer, in the case where the gene has many loci but the number of genotypes is extremely low.

4. Fourth embodiment

According to a preferable embodiment of the present invention, the method mentioned above can be used to check whether each of genes of a cancer cell is placed in expression conditions or not. Compared to the first embodiment mentioned above, the third embodiment may be a so-called “inverse implication”.

First, operational nucleic acids representing various logical equations are prepared. Each of the operational nucleic acids has terms of a logical equation, namely, DCNᵢ, DCNᵢ*, S, and M, whose 5′ ends are individually phosphorylated. In addition to these operational nucleic acids, a complementary nucleic acid 9 for linkage as shown in FIG. 22 is prepared. The complementary nucleic acid 9 for linkage (ligation complementary nucleic acid) is used for ligating the terms of the logical equation. It is desirable that the ligation complementary nucleic acid 9 contain sequences complementary to two portions sandwiching a partition portion to be ligated. These nucleic acids are hybridized to each other and ligated with ligase, it is possible to obtain an operational nucleic acid in which the terms of logical equation are connected to each other. If the sequence of the ligation complementary nucleic acid 9 is designed carefully, it is possible to prevent an improper logical equation from being formed.

The inverse implication is solved by using the operational nucleic acid as follows. First, cDNA is taken from a cancer cell and converted into sequences corresponding to terms of logical equation in accordance with the first embodiment. A logical operation is performed by using operational nucleic acids representing various logical equations prepared in advance. Finally, of the logical equations expressed by these operational nucleic acids, a satisfactory logical equation is detected. If there is the logical equation of the operational nucleic acid gives 1, the contents of the operational nucleic acid are analyzed and interpreted. In this way, it is possible to detect which gene is placed under an expression condition or a nonexpression condition. At least, the condition whether a gene is expressed or not can be partly known.

The sequence of an operational nucleic acid arbitrarily prepared is identified as follows. First, the reaction of the operational nucleic acid is performed in a single container. Then, the operational nucleic acid is recovered by a streptoavidin magnetic beads via a marker oligonucleotide attached with a biotin molecule according to the first embodiment. Subsequently, the operational nucleic acid molecule is subjected sequencing. In this manner, the contents of the operational nucleic acid can be read out, whereby a logical equation can be read out. Alternatively, an arbitrary operational nucleic acid may not be prepared. In this case, first, a logical equation is determined and then, the operational nucleic acid is synthesized in accordance with the method of preparing the operational nucleic acid by ligation. In practice, logical equations may be fixed on a DNA microarray by addressing them and the logical equations are identified at the time of detection. Alternatively, nucleic acids of the same type are placed in a single container to react them.

If these results of genes taken from a cancer cell are compared to those from a normal cell, it is possible to readily identify genetic diseases caused by combination of unknown genomic nucleotide sequences and casual genes of diseases such as cancers caused by abnormality of genes produced by combination of unknown gene expressions.

5. Consideration

In the prior art, there is neither a technique nor an idea for converting the information “a specific nucleic acid is absent” into a nucleic acid expression. To detect the expressional state of mRNA, a DNA microarray is often used. In the DNA microarray, probes such as oligo DNAs, which have been designed based on the nucleotide sequence of a known gene, or cDNAs previously obtained, are fixed on a slide glass in the form of an array.

The gene-expression is detected on the microarray as follows. First, cDNA is prepared from mRNA. The cDNA is labeled with a fluorescent molecule and allowed to hybridize with a probe on the microarray. As a result, the cDNA with a fluorescence label bonds to a site at which a specific probe is fixed. Since fluorescence is emitted from the site, the site can be detected. However, when mRNA (gene) is not expressed, cDNA labeled with fluorescence cannot be prepared. This means that it is impossible to detect that the gene is not expressed. Unfortunately, mRNA happens to disappear during the experimentation in some cases. In other cases, it sometimes falsely determined that a gene is not present, even though the gene is expressed due to a low amount of fluorescent-labeled cDNA. Unlike such a conventional method mentioned above, the information that a gene is not expressed can be visualized in the method according to an embodiment of the present invention.

When nucleic acids of a gene are reacted, if numerous genes are copresent, nucleic acids make a hybrid in an unexpected combination. The structure of a double stranded nucleic acid can be stabilized if nucleic acids such as guanine and cytosine are contained in a larger number in the nucleotide sequence. However, the number of such nucleic acids varies depending upon the nucleic acids of a gene to be treated, the most suitable hybridization temperature differs. As a result, not all nucleic acids make an appropriate hybrid without a mismatch. In another case where there is a nucleic acid having a nucleotide sequence which takes a stable form within the molecule in a reaction mixture, such a nucleic acid lowers the reactivity with a target sequence. Therefore, the hybridization reaction may not proceed as expected in theory. Unlike such a conventional method, in the method according to the present invention, the reaction can be stably performed. This is because, in the present invention, information is first converted into the nucleic acid molecule designed under preferable conditions and then subjected to the reaction.

On the other hand, in a method using an enzyme associated with chemical luminescence, detection can be made accurately. However, the operation for detecting the chemical luminescence is nuisance and consumes much time. In addition, it is difficult to perform a plurality of chemical luminescence reactions in a single tube. On the other hand, when an operation is performed by use of an operational nucleic acid which gives the results based on a single color or luminescence, only one type of operational nucleic acid is reacted in a single tube. In contrast, according to an aspect of the present invention, it is possible to detect numerous target nucleic acids simultaneously and accurately.

In the conventional method, a logic equation which has been prepared only on the precondition that a nucleic acid is present, into an operational nucleic acid. However, problems usually present in the real world is a so-called “inverse implication”. To be more specific, when genes are checked for their expression state in a genetic disease, which nucleic acid of a gene is present or absent when the genetic disease occurs is a matter of concern. Accordingly, what is important is to obtain a logical equation as to the presence or absence of the nucleic acid of the gene. Up to present, which gene is present or absent has been solved by subjecting data obtained from a DNA micro array to a cluster analysis using a large-scale computer. Therefore much time and cost are required for the cluster analysis. However, if the present invention is employed, it is possible to solve such a problem economically in a short time.

In this text, a gene analysis method is described. However, the present invention enable to perform information processing by use of various type of parallel operations, within the scope of the present invention. In other words, it would be obvious to a person skilled in the art to understand that if the present invention is applied to parallel computation of data for solving mathematical problems other than gene analysis, excellent advantages can be obtained.

II. Molecular operation apparatus

1. Outline

According to a preferable aspect of the present invention, there is provided a computer for executing the information processing method mentioned above. More specifically, the present invention provides a molecular computer comprising an electronic operation section and a molecular operation section. The electronic operation section substantially controls the function of the molecular operation section. In the molecular operation section, an operation is performed by using molecules under the control of the electronic operation section.

In the computer of the present invention, the operation is basically performed as follows. A calculation is first performed by use of parallelism of the molecules and then, a logical equation is evaluated on the basis upon the data thus obtained, by an electronic computer as conventionally used, at a high speed. The computer disclosed in the present invention makes it possible to efficiently perform the parallel operation of data and gene analysis by using nucleic acids in an operation. Furthermore, data and programs are expressed by nucleic acid molecules and commands are executed by molecular reactions in the present invention. It is therefore possible to realize an extraordinarily large memory capacity and achieve a high throughput of parallel processing, compared to conventional electronic computers.

To develop the computer of the present invention, first, the present inventors found out that the content of an operation program (to be used in a molecular computer) must be changed into the form of data which can be recognized and executed by a molecular operation section. More specifically, they found it preferable that a molecule is converted into a coding molecule attached with a specific code, and then variables and constants in the operation program are automatically converted into coding molecules before the computation is performed in the molecular operation section. Furthermore, they found that such a conversion operation is preferably performed in the electronic operation section to improve an operation speed.

In other words, in the molecular computer of the present invention, the molecular operation section and electronic section complementarily share their functions with each other. Therefore, the molecular computer of the present invention is easy to use. In addition, the molecular computer can solve the NP-complete problem at a high speed. More specifically, the computer of the present invention overcomes disadvantages of the molecular operation section: low speed of operations such as inputting and displaying letters, and simple four fundamental operations of arithmetic, by using the electronic operation section, thereby improving the operation speed. In other words, the computer of the present invention is a hybrid molecular computer in which the electronic operation section is responsible for a high-speed operation attained by an electronic computer at a high speed, and the molecular operation section takes charge of other functions.

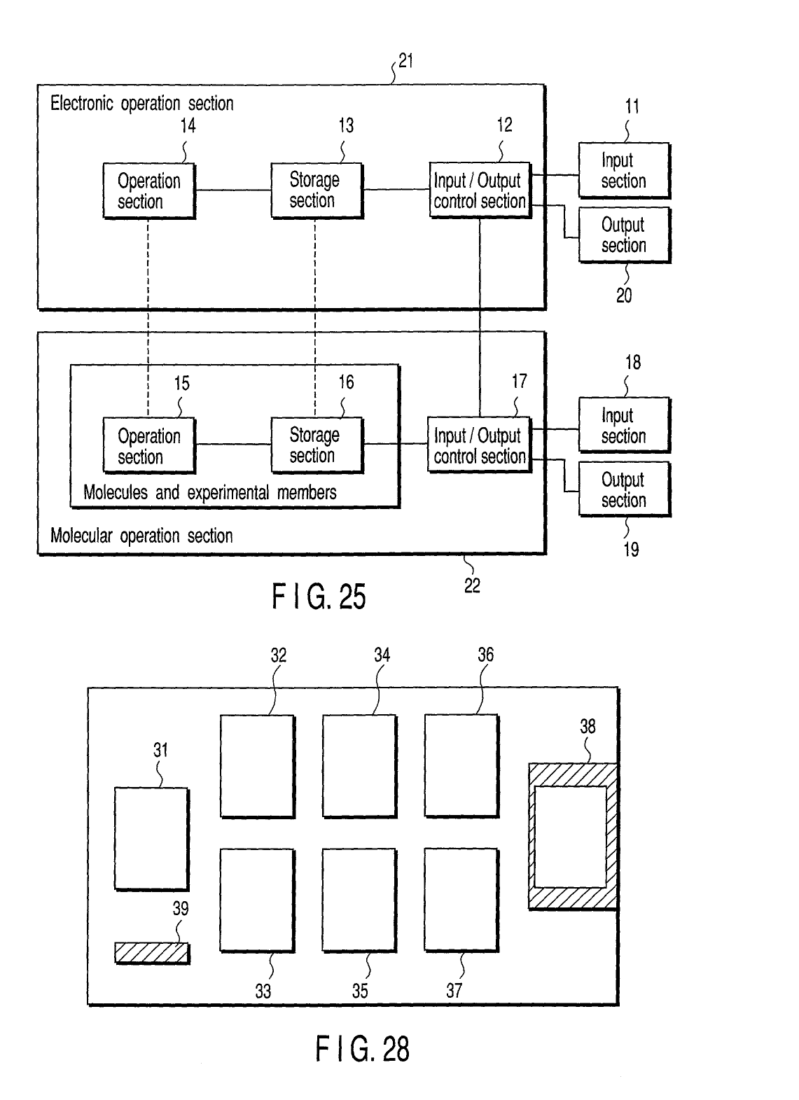

FIG. 25 shows a basic computer of the present invention. The computer comprises an electronic operation section 21, input section 11 and output section 20 to the electronic operation section 21, a molecular operation section 22, an input section 18 and an output section 19 to the molecular operation section 22. The electronic operation section 21 at least has an operation section 14, storage section 13, and input/output control section 12. Furthermore, the electric operation section may have structural elements of a general electronic computer.

The molecular operation section 22 has an operation section 15, storage section 16 and input/output control section 17. As the operation section 15 and the storage section 16, molecules and experimental materials are actually used. The sections 15 and 16 simultaneously attain operation and data storage.

In the computer of the present invention, the electronic operation section 21 basically controls the molecular operation section 22. First, a desired program is input into the electronic operation section 21. Based on the input data, a plan for molecular operation to be performed is prepared in the molecular operation section 22. Based on the molecular operation plan thus prepared, required molecules are designed. The obtained data are sent to the molecular operation section 22, in which molecular operation is performed. The plan of the molecular operation may be prepared by searching a table, in which input data stored in the storage section 13 are listed in correspondence with molecular operation to be designed, by the operation section 14 (such as CPU), and selecting the operation corresponding to the input data. Alternatively, the molecular design may be performed by searching the table by the operation section 14 (such as CPU) and selecting the corresponding molecule.

Furthermore, the output of the operation obtained by the molecular operation section 22 may be returned to the electronic operation section 21, in which a desired processing may be performed by the operation section 14 and a processing means arbitrarily equipped. The results are summed up and operated in accordance with the processing form previously input and stored in the storage section 13, and finally output into the output section 19 or 20 in a desired form. During these processing operations, the states of the operation section and the storage section of the molecular operation section can be indirectly monitored via the input/output control section in the electronic operation section. This manner is indicated by a broken line in FIG. 25.

On the other hand, the molecular operation section 22 is responsible for experimentally synthesizing a nucleic acid molecule and executing desired molecular operations by use of the synthesized nucleic acid molecules. The examples of molecular operation performed in the molecular operation section will be described later.

The operation section 14 of the electronic operation section 21 is responsible for converting an initial value input from the input section 11 into a coding molecule, converting the procedure and the function of an operation program into an operation reaction of coding molecules, and preparing an operation manual of the operation reaction from the operation program stored therein. The electronic operation section 21 controls the following assignments: the assignment of molecules to variables, assignment of experimental containers, assignment of experimental members (for example, a pipette tip), and the arrangement of reagents, assignment of transfer and operation of experimental tools (such as container, pipette tip), setting of the temperature control of a thermal cycler, and the determination of an implementation sequence of an experiment.

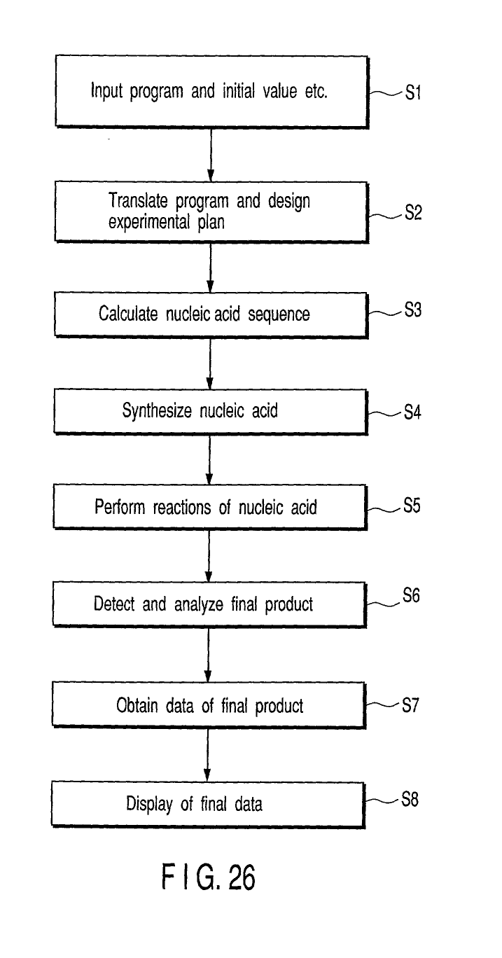

A basic operation manual of a computer will be explained in accordance with a process flow shown in FIG. 26.

(S1) A desired operation program is input from the input section 11 of the electronic operation section 21. Data such as constants and variables of the operation program are stored in the storage section 13.

(S2) Individual initial values of the data stored in the step S1 are converted by the operation section 14 into coding molecules in accordance with the corresponding data previously stored in the storage section 13. The operational function of the operation program is converted into an operation reaction of the corresponding coding molecules. Furthermore, according to operation program stored in the memory 13, the implementation manual of the operation reaction is prepared. In the step S2 herein, each of the conversion operations is called “program translation”, and the preparation of the implementation manual is also called “set-up of experimental plan”.

(S3) In the operation section 14, a nucleic acid sequence actually used is designed from the coding molecules obtained in the step of S2. Note that this step may not be performed if the nucleic acid sequence previously designed is used.

(S4) Data obtained hereinabove in the electronic operation section 21 is sent to the molecular operation section 22, in which a coding molecule, that is, a nucleic acid having the sequences designed in the step (S3) is synthesized in the operation section 15. Materials required for the nucleic acid synthesis are supplied form the input section 18. This synthesis step is not required if the nucleic acid previously designed and synthesized is used. In this case, the nucleic acid synthesized in advance is input from the input section 18.

(S5) An operation reaction is performed in accordance with a program by using the operation molecule prepared in the step (S4) and in accordance with the implementation manual obtained in the step (S2).

(S6) The reaction product obtained through the operation reaction performed in the step (S5) is detected and identified. In this way, the reaction product is analyzed.

(S7) The data of the reaction product obtained in the step (S6) is sent to the electronic operation section 21. An information processing program which has been stored in the electronic operation section 21 is read out. Based on the information processing program read out, the data of the reaction product obtained from the molecular operation section 22 is processed to obtain final data.

(S8) The final data obtained in the step (S7) is processed so as to accord with the output formula previously stored in the storage section 13 and output from the output section 20.

If desired, the aforementioned steps may be carried out by a loop operation. For example, the results obtained in each step are compared to the conditions previously stored in the storage section 13. Depending upon the comparison results, the previously performed steps alone or in combination may be repeated by a loop operation.

Furthermore, if it is difficult to perform the steps automatically or if the automatic operation requires a large-scale device, for example, operations, such as cloning culture and colony picking, are desirably performed by hands, a nucleic acid is once output into the output section 19, and then, the experimental operation to be performed is displayed in the output section 20. After the experimental operation is manually performed, the nucleic acid is again input into the input section 18. The turn-on is instructed through the input section 11. Alternatively, the input may be automatically detected by the input section 11, to start the computation.

In the case where an operation program is directed to obtaining a desired molecule and therefore, the operation results need not to be displayed, the computation process may be completed at the step S5 which provides the desired molecule. The program may directly go to the step 8 without performing steps S6 and S7. In this way, “completion of calculation” may be displayed in the step S8. A representative example of this case is where an operation can be performed in order to select an oligo DNA for specifically detecting a specific gene.

Conversion to coding molecules in the step S2 can be performed by reading out a molecule conversion table previously stored in the storage section, searching and picking up the corresponding data included in the molecule conversion table. A preferable molecule conversion table include coding molecules to be used molecular operation and data to be input in the electronic operation section, both corresponding with each other. More preferably, the molecular conversion table used herein may be an electronic data version of the one-to-one correspondence table generally used.

The operation procedure of the operation program and the conversion of a function into an operational reaction are performed by reading out the procedure-conversion table stored in the storage section and searching and picking up the corresponding data. In the procedure conversion table, the steps of molecular operation actually carried out, the order of the steps to be carried out, and repeat number of the steps are brought to correspond and data to be input to the electronic operation section may be brought into correspondence with data to be input into the electronic operation section. More specifically, use is made of an electronic version of a generally-used one-to-one correspondence data.

The data obtained in each of the steps may be stored in the storage section 13 per step, partly or in its entirety.

As the input section of the electronic operation section 21 of the computer of the present invention, a manual input means, such as a keyboard and a mouse, may be used. As the output section of the electronic operation section of the computer of the present invention, a general output means such as a display or a printer may be used.

In the aforementioned manual, a nucleic acid molecule is synthesized in the operation section 15 arranged in the molecular operation section 22. However, the operation section may be arranged outside the molecular operation section 22 and the electronic operation section 21. In this case, the operation section may be arranged as a molecular synthesis section which is connected to the electronic operation section 22 and the molecular operation section 21. To carry out an operation reaction using nucleic acid molecules, the operation section 15 within the molecular operation section 22 may comprise the following portions and means: a reaction portion for performing various reactions, a nucleic acid holding portion for holding nucleic acids required for each of the reactions, a reagent holding portion for holding a reagent and a buffer required for each of the reactions, an enzyme holding portion for holding an enzyme required for each of the reactions, a heating means for heating each of the portions if desired, a cooling means for cooling each of the portions if desired, a dispensing means such as a dispenser pipette, a washing means for washing a tool such as a pipette and a reaction container, and a control means for controlling each of operations. These portions and means may be used all or in combination of some.

The operation section 15 may comprise a detection portion with a detection means for detecting and identifying a reaction product produced by a desired reaction. However, the detection portion may not be necessarily included in the operation section 15. The size of the detection portion may be increased depending upon the detection means to be used. In this case, the detection portion may not be arranged within the electronic operation section 21 and the molecular operation section 22, and arranged as an independent portion which are connected to the electronic operation section 21 and the molecular operation section 22.

The detection using the computer of the present invention may be carried out as follows. A reaction product produced by an operation reaction is electrophoresed and the position of a peak is detected to determine its length. In this manner, the nucleic acid molecule can be determined. Alternatively, the reaction product is subjected to DNA sequencing. The DNA sequence of the reaction product is determined based on the data obtained by the DNA sequencing. Based on the sequence obtained, corresponding coding nucleic acids initially assigned may be determined. As another method, if a DNA micro array and a scanner are equipped, the sequence of the coding nucleic acid hybridized may be read out by the scanner.

In the aforementioned flow chart, the detection results are sent to the electronic operation section 21 and output from the output section 20. However, molecules obtained as the detection results in the molecular operation section 22, may be obtained as it is, as raw data.

To design a series of experimentation manuals (experimentation sequence) in the electronic operation section 21, it is necessary to determine the experimentation sequence disambiguous determined from the input program, problem, and an initial value. For example, in the case of a 3SAT program, the experimental design may be determined by developing the loop of the program and determining a branch condition in advance with reference to the problem. Therefore, a whole experimental design can be determined at the time the operation of the program is initiated. However, depending upon the program to be used, a sequence detection operation step is inserted in the middle of a loop to confirm the sequence and then conditional branch must be performed in some cases. In this case, after the detection operation is performed, the experimental plan of the following step may be designed depending upon the results of the detection operation.

In the computer of the present invention, the experimental operation, for example, cDNA synthesis, is expressed by a function and stored in the electronic operation section in connection with the experimental operation. By virtue of these preparation, an automatic operation can be performed. If the coding reaction is automatically performed, each process, for example, logical operation and/or sequencing can be automatically performed in parallel.

According to the present invention, various working robot systems for use in conventional experimentations, clinical trials, and manufacturing processes, may be employed. In the present invention, the robots may be operated so as to correspond to working plans for preferentially attaining an object without being limited by the program for the electronic operation section prepared in advance. In some cases, the most suitable working plan may be incorporated automatically into a robot system.

For example, when a human gene is converted into the coding sequence, it is preferable to use a partial sequence of a foreign organism such as a virus having a low homology of nucleotide sequence to the human gene. In the case where an abiotic problem such as 3SAT is solved, sequences having substantially an equal melting temperature (Tm) and causing no cross hybridization are arbitrarily designed and used the coding sequences. If many coding sequences are required, it is preferable to use orthonormal sequences obtained through calculation performed in the electronic operation section 21 so as to satisfy desired conditions.

Since an operation can be performed by directly inputting molecules, if the present invention is used in, for example, gene analysis, experimental errors may be minimized. To explain more specifically, since a nucleic acid is converted into a coding nucleic acid, and then, the coding nucleic acid can be operated as it is in the computer, an experimental error can be minimized. If the computer of the present invention is used, the computation time and running cost can be saved. The gene analysis can be programmed, the experimentation can be performed while the operation is automatically performed. In addition, the expressed gene is subjected to coding OLA (Oligonucleotide Ligation Assay) to convert into coding nucleic acids, which further subjected to PCR amplification by using common sequences present at the 3′ and 5′ ends of the coding nucleic acid. As a result, an expression ratio can be accurately detected. The coding OLA will be more specifically explained later.

2. Detailed expression

(1) Computer

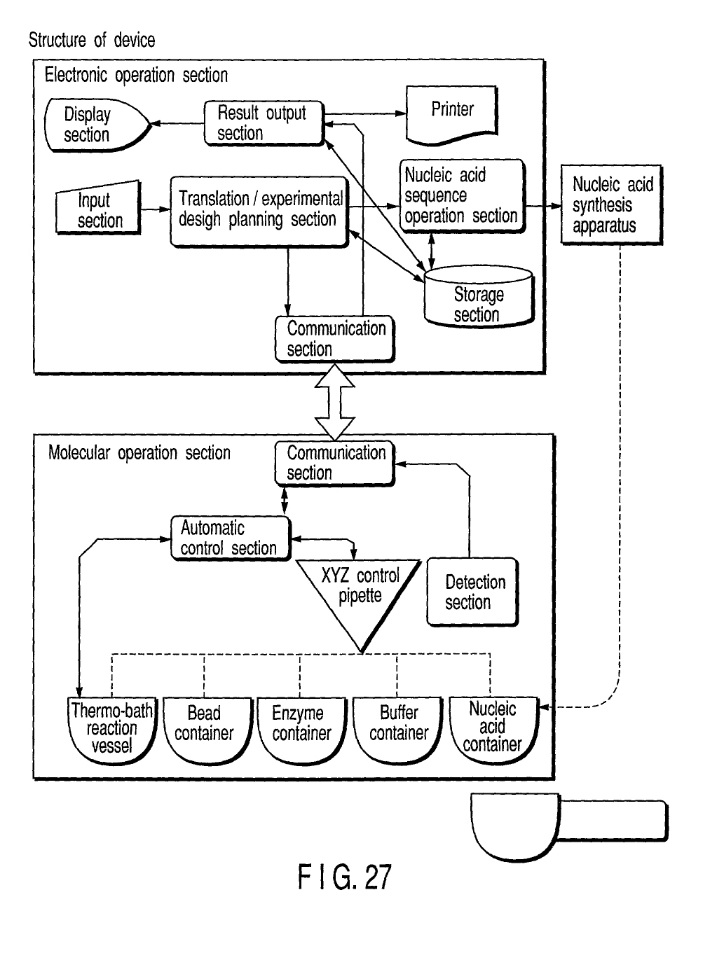

Now, the present invention will be more specifically explained below. FIG. 27 shows an example of the computer of the present invention, which consists of an electronic operation section, a molecular operation section, and a nucleic acid synthesizing portion. These three portions are mutually connected.

An example of an operation manual in the electronic operation section will be explained. An operation program and an initial value input from the input section are stored in a storage section. Alternatively, before being stored in the storage section, they are subjected to a translation/experiment design planning section, in which they are converted into coding molecules and an operation reaction of the coding molecules. At the same time, the manual as to how to perform the operation reaction is prepared. Thereafter, the coding molecules and the operation manual are stored in the storage section. As a next step, in the nucleic acid sequence operation section, an operational molecule is calculated and designed on the basis of data (after converting into the operation reaction) obtained in the above. The data obtained in these steps may be displayed on the display portion by way of a result output section. Alternatively, the computer may be set such that desired data alone is displayed any time. The desired data may be printed out if desired.

The nucleic acids such as operational molecule (required for molecular operation) obtained in the nucleic acid sequence operation section are synthesized on the basis of the data obtained by the operation in the electronic operation section. The nucleic acid synthesized is transferred to a nucleic acid container of the molecular operation section.

On the other hand, the molecular operation section and the electronic operation section are equipped with a communication section for receiving or sending information from or to the electronic operation section. The communication may be attained by a cable communication means or a wireless remote communication means such as electronic wave.

In the electronic operation section, a thermo-bath reactor for performing a reaction therein, a bead container for storing beads, an enzyme container for storing an enzyme, a buffer container for storing a buffer, and a nucleic acid container for storing a nucleic acid. The containers may be a beaker, test tube, microtube, and the like. Alternatively, other storing means generally used may be used.

As the thermo-bath reactor, any reactor may be used as long as it is capable of adjusting the temperature of the container. The thermo-bath reactor is constructed by fitting a reaction vessel to a generally used thermo-bath. In this case, beads are used as a carrier. However, another carrier may be used. When another carrier is used, an appropriate structural element may be used in place of or in addition to the bead container. The enzyme container may be set together with a temperature controlling means such as a cooler to protect an enzyme from being inactivated. If necessary, a temperature controlling means such as a cooler or heater may be arranged at other storing means.

The reaction is performed in the thermo-bath reactor in accordance with an operation program. For example, a desired amount of the content is taken from a storage container by an XYZ control pipette, and then the reaction is performed under conditions including a desired temperature. The operation of each section is controlled by an automatic operation section within the molecular operation section. The operation of the automatic operation section is controlled by the electric operation section. The XYZ control pipette used herein is a pipette which can be moved, if necessary, in the XYZ direction and/or upward and downward, under the control of the automatic control section.

After completion of the reaction, a reaction product is detected and identified at a detecting section. As the detecting section, any measuring means or analysis means such as an electrophoretic device, sequencer, chemical luminescent measurer, or a fluorescent measurer may be used as long as it is used for detecting or analyzing a nucleic acid molecule.

In the present invention, the molecular operation section at least includes a means to be used in the first to final steps of a biological specific reaction including a hybridization reaction, enzymatic reaction, antigen-antibody reaction. The initiation point of the reaction at least includes, at earliest, a step in which a reaction pair significantly exhibits selectivity, and, at latest, the step immediately before an object such as detection or isolation is attained. Furthermore, the completion point of the reaction at least include, at earliest, the most earliest point of the step in which detection or isolation is attained, and include, at latest, the time point exceeding the point at which the detection and isolation is fully attained.

FIG. 28 shows an arrangement of the molecular operation section of the present invention. In the molecular operation section, it is possible to arrange a 8-chip pick-up rack 31, a single-chip pick-up rack No. 0, 32, a single-chip pick-up rack No. 1, 33, a 96-hole multi titer plate No. 2 (MTP), a 96-hole MTP No. 0, 35, a 1.5 mL tube rack 36, a 96-hole MTP No. 1, 37, a thermal cycler 96-hole MTP 38, and a chip discharge hole 39. However, the arrange of the molecular operation section is not particularly limited thereto and can be modified in various ways, if necessary.

(2) Molecular operation in the molecular operation section

The molecular operation in the molecular operation section of the molecular computer according to an aspect of the present invention, may be the same as the aspect of the present invention described in the paragraph (I) mentioned above.

First, we will describe a problem residing in a DNA computation based on a conventional Adleman-Lipton paradigm. A first problem resides in that it is necessary to form a pool for storing DNA molecules expressing all potential solutions. This is because the number of potential solutions of the NP complete problem exponentially increases compared to the number of variables. Therefore, if the size of the problem increases, it is impossible to form a pool for storing all possible solutions even if a DNA computer having a super memory capacity is used. This is a significant problem associated with the Adleman-Lipton paradigm.

Now, with respect to a typical NP complete problem, that is, the satisfiable problem (3SAT) of “three product-of-sum logical equation”, will now consider. Assuming that the number of logical variables is 100, the number of possible solutions will be 2¹⁰⁰=1.3×10³⁰. If a single variable is expressed by a DNA molecule having 15 base pairs, at least 20,000 tons of DNA molecules are required in order to prepare a complete probe. The amount of 20,000 tons is a unrealistic value. In this case, if a single solution is expressed by a single molecule, the risk of losing the single molecule in the middle of an operation reaction is high. Therefore, in practice, a larger amount of DNA molecules is actually required. Assuming that the number of variables is 200, The volume of the DNA molecules required is about 40 trillion-times of the mass of the Earth. It is virtually impossible to solve the problem.

Under the circumstances, the inventors assumed that the NP complete problem may be solved by executing an algorithm based on a dynamic programming by a molecular computer (also called as DNA computer). In place of preparing all possible solutions in the beginning as the Adleman-Lipton paradigm, possible solutions for a part of the problem are prepared. From the possible solutions, a right solution is selected and extracted. This procedure is repeated while the size of the part of the problem gradually increases, and finally, the solution of the original problem can be obtained. If this technique is used, the number of possible solutions to be prepared can be greatly reduced.

In the paragraph (1,1) “preparation”, mention is made of a design of a molecule. As a first step, an operation program is converted into requisite data in the translation/experiment design planning section of the electronic operation section. Thereafter, a nucleic acid sequence suitable for desired conditions in the nucleic acid sequence operation section. Note that the first step is performed in the operation section of the electronic operation section. A nucleic acid is synthesized based on the data obtained in the electronic operation section. Molecular operation is carried out in the molecular operation section.

(3) Program

3SAT, a typical example of the NP complete problem, can be solved by performing a DNA computation in accordance with the following program based on an algorithm of dynamic programming.

Equation 2

Problem: 4 variables, 10 clauses

(x₁ [symbol] x₂ [symbol] [symbol] x₃) [symbol] (x₁ [symbol] [symbol] x₂ [symbol] [symbol] x₃) [symbol] ( [symbol] x₁ [symbol] x₂ [symbol] x₃) [symbol] ( [symbol] x₁ [symbol] [symbol] x₂ [symbol] x₃) [symbol] (x₁ [symbol] x₃ [symbol] x₄) [symbol] ( [symbol] x₁ [symbol] x₂ [symbol] x₄) [symbol] ( [symbol] x₁ [symbol] [symbol] x₃ [symbol] x₄) [symbol] (x₂ [symbol] [symbol] x₃ [symbol] [symbol] x₄) [symbol] (x₂ [symbol] x₃ [symbol] [symbol] x₄) [symbol] ( [symbol] x₂ [symbol] x₃ [symbol] [symbol] x₄)

where a symbol “ [symbol] ” is a logical AND, a symbol “ [symbol] ” is a logic OR, a symbol “ [symbol] ” denotes negation.

Equation 3

Solution:

YES

{x₁,x₂,x₃,x₄}={1.1.1.1}

Using the computer, 3SAT of 4 variables, 10 clauses, was solved. The problem which has been solved is shown in Equation 2. The solution of the problem is shown in Equation 3. The problem of Equation 2 consists of x₁, x₂, x₃ and x₄ variables. 10 of clauses consisting of 3 literals are coupled by a logical OR. Solving the equation is to obtain a set of values satisfying the equation, more specifically, to know whether a solution is present or not. If present, a set of values is obtained.

Equation 4

function dna 3 sat (u₁, v₁, w₁, u₂, v₂, w₂, u₃, v₃, w₃, . . . , u₁₀, v₁₀, w₁₀) begin

T₂ = {X₁^{T}X₂^{T}, X₁^{F}X₂^{T}, X₁^{T}X₂^{F}, X₁^{F}X₂^{F}};
for k = 3 to 4 do

amplify (T_{k - 1}, T_{W}^{T}, T_{W}^{F});
for j = 1 to 10 do

if wⱼ = xₖ then

T_{W}^{F} = getuvsat(T_{W}^{F}, uⱼ, vⱼ);

end
if wⱼ = ┤xₖ then

T_{W}^{T} = getuvsat(T_{W}^{T}, uⱼ, vⱼ);

end

| end | |
|---|---|
| T^{T} = append (T_{W}^{T}, Xₖ^{T}, Xₖ₋₁^{T / F}); | T^{F} = append (T_{W}^{F}, Xₖ^{F}, Xₖ₋₁^{T / F}); |

Tₖ = merge (T^{T}, T^{F});

end
return detect (T₄);

function getuvsat (T, u, v)
begin

| | |
|---|---|
| Tᵤ^{T} = get (T, + Xᵤ^{T}); | Tᵤ^{F} = get (T, − Xᵤ^{T}); |

| | |
|---|---|
| T′ᵤ^{F} = get (Tᵤ^{F}, + Xᵤ^{F}); | /* can be omitted * / |

Tᵥ^{T} = get (Tᵤ^{F}, + Xᵥ^{T});
T^{T} = merge (Tᵤ^{T} , Tᵥ^{T} );
return T^{T};

end

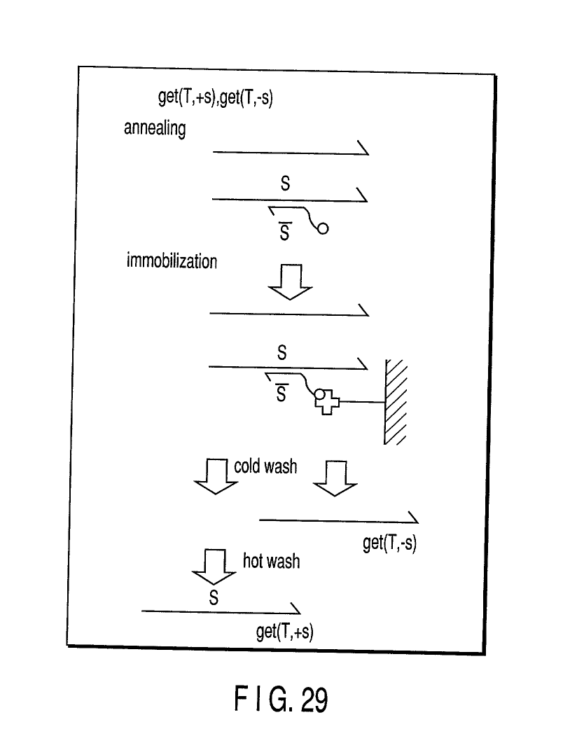

To solve the given problem, the function represented by Equation 4 is executed by the computer of the present invention. The notation is made in conformance with PASCAL language. Function dna3sat is a main function to solve a problem when a solution is present. In Function dna3sat, Function getuvsat is included. Functions “dna3sat” and “getuvsat” consist of 4 basic Functions: “amplify”, “append”, “merge”, and “detect”. These functions are executed by DNA reactions shown in FIGS. 29 to 32, respectively.

Next, the reaction of each basic Function will be explained. First, Functions get (T, +s) and get (T, −s) will be explained in accordance with FIG. 29.

The “get” is a function for obtaining a single-stranded oligonucleotide containing s sequence or a single-stranded oligonucleotide containing no s sequence from a solution mixture T (tube) of oligonucleotides. The symbol s used herein represents a specific sequence consisting of several nucleotides or several tens nucleotides. The “get (T, +s)” represents getting an oligonucleotide including the s sequence. The “get (T, −s)” represents getting an oligonucleotide including no s sequence. In a first step, an oligonucleotide having a complementary sequence to the s sequence and labeled with biotin at the 5′ end is placed in a tube T, in which it is annealed with an oligonucleotide having the s sequence.

The resultant hybrid is captured by magnetic beads having streptoavidin attached on the surface. Thereafter, the magnetic beads is washed with a buffer solution (cold wash) at a temperature at which the hybrid having a complementary s sequence is not dissociated. As a result, the oligonucleotide having no s sequence can be obtained from the buffer solution. In this manner, Function get (T, −s) can be executed.

After completion of the cold wash, the beads are washed with a buffer solution of a relatively high temperature at which the hybrid can be dissociated. As a result, the oligonucleotide having the s sequence can be obtained. In this manner, the get (T, +s) can be executed. As described above, two functions can be executed by a single operation.

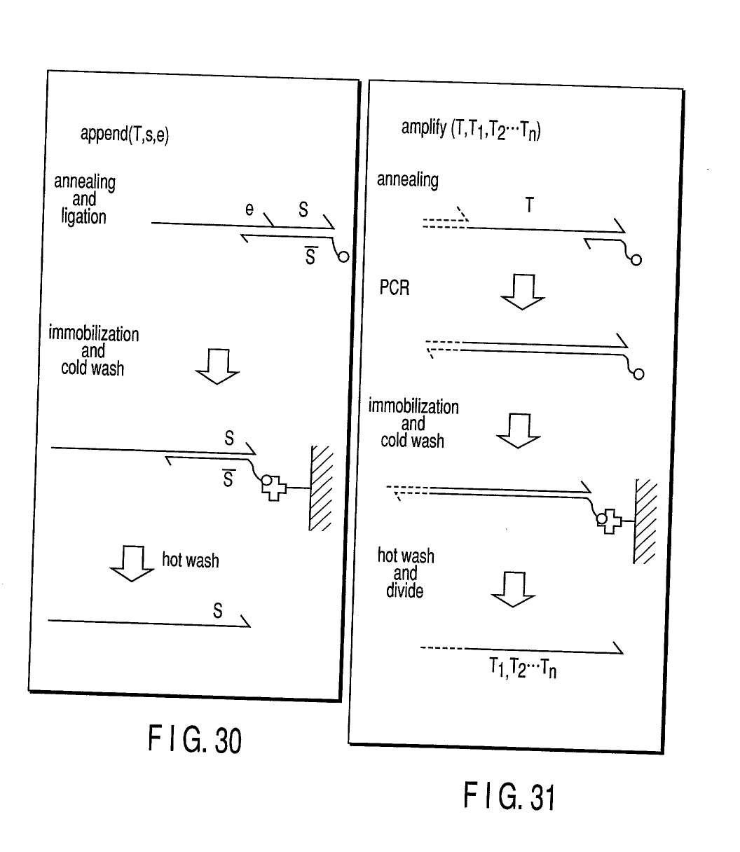

The reaction of “append” (T, s, e) will be explained in accordance with FIG. 30. First, in a solution mixture T of oligonucleotides, an oligonucleotide having the s sequence is ligated to the 3′ end of a single stranded DNA having an e sequence at the 3′ end. In this manner, a single stranded oligonucleotide having the s sequence ligated thereto can be obtained. Function “append” is thus executed. The s sequence and e sequence are specific sequences consisting of several nucleotides or several tens of nucleotides, as mentioned above. In this reaction, the following oligonucleotide is used. The oligonucleotide of the s sequence whose 5′ end is phosphorylated. The ligation oligonucleotide, whose 5′ end is labeled with biotin, and in which a sequence complementary to the s sequence (at the 5′ end) and a sequence complementary to the e sequence (at the 3′ end) are arranged next to each other. These oligonucleotides are placed in a tube T and a reaction is initiated. The ligation oligonucleotide is hybridized with a target oligonucleotide e sequence and oligonucleotide s sequence to make hybrids. The hybridization reaction is performed at a relatively high temperature so as not to make a hybrid with a mismatch. Subsequently, the hybrids are ligated by using Taq ligase having an activity at a high temperature. Thereafter, the resultant hybrid is captured by streptoavidin magnetic beads and washed (hot wash) at a high temperature at which the hybrid is dissociated. As a result, an oligonucleotide having the s sequence ligated at the 3′ end can be recovered.

Next, according to FIG. 31, the reaction of Function “amplify” (T, T₁, - - - Tₙ) will be explained. A oligonucleotide contained in the reaction solution T is amplified by a PCR reaction. The amplified double stranded oligonucleotide is dissociated into single stranded oligonucleotides and divided them into reaction solutions of T₁ to Tₙ. In this embodiment, the oligonucleotide contained in the reaction solution T has a common sequence at both ends for use in amplification. All oligonucleotides contained in the solution T can be amplified by using a set of primers. Of the primers, the 5′ end of a primer having a complementary sequence to the 3′ end of a target oligonucleotide, is attached with biotin. The oligonucleotide in the solution T is amplified by the common primer and captured by streptoavidin magnetic beads. The oligonucleotide captured by beads is washed at a high temperature such as 94° C. so as to completely dissociate the double strand. As a result, the strand originally contained in the solution T can be extracted into a buffer solution. The extracted solution may be divided into reaction solutions of T₁ to Tₙ.

Function “merge” expresses an operation for integrating a plurality of solutions serving as arguments into one.

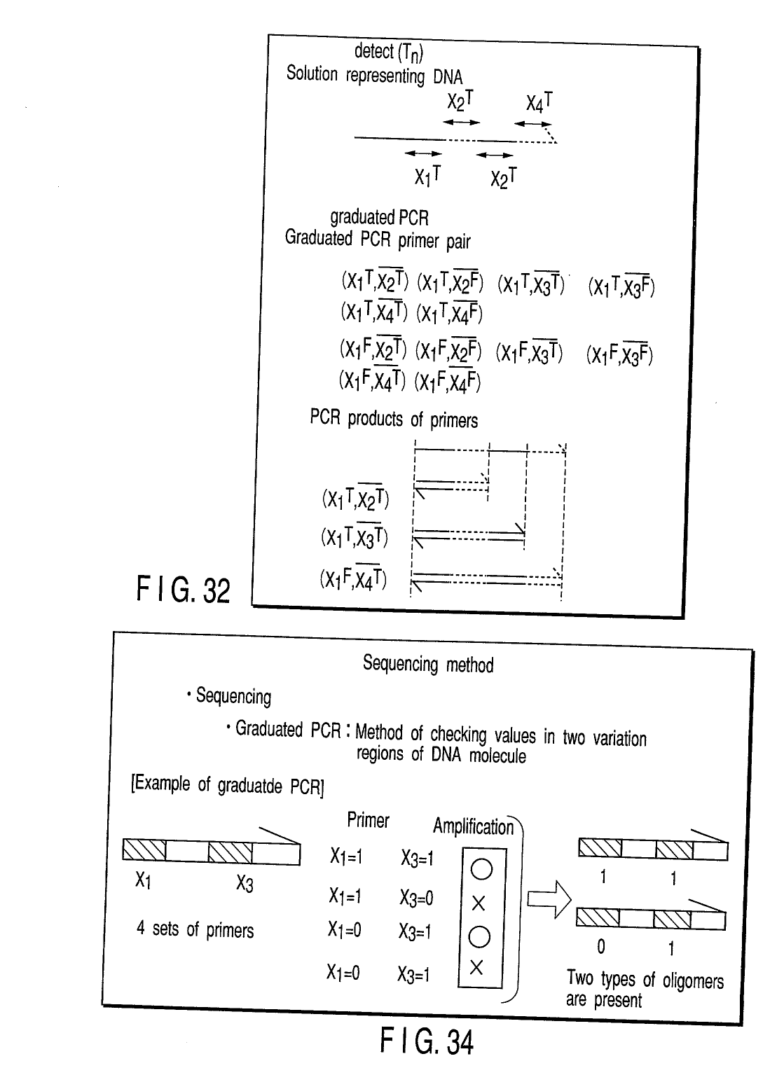

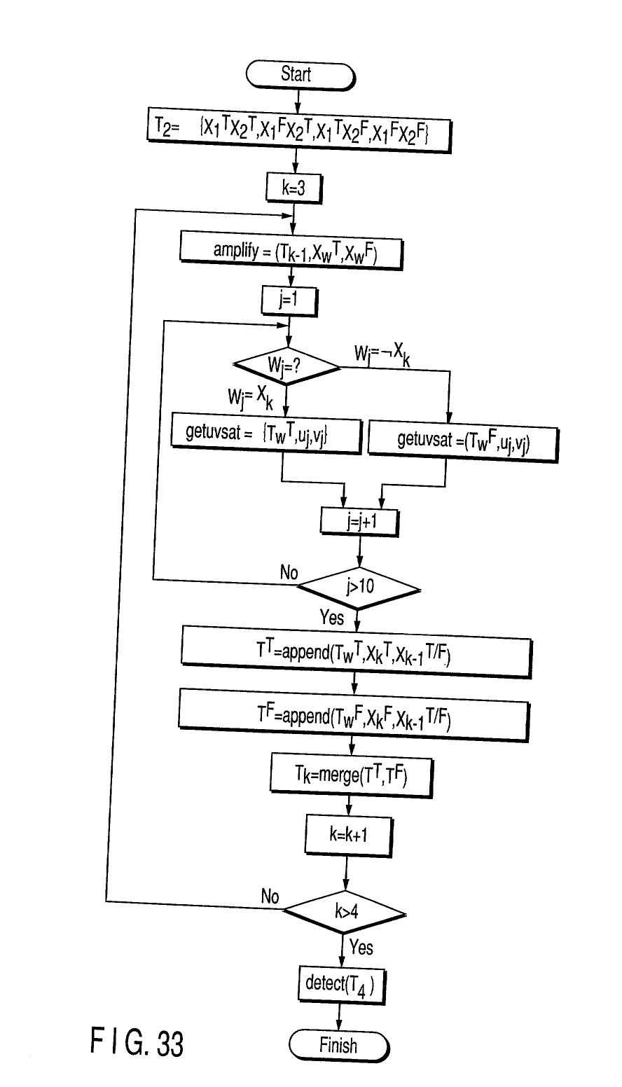

Finally, Function “detect” will be explained in accordance with FIG. 32. The “detection” can be executed by manually performing a reaction. Now, a method of executing Function “detect” by the computer of the present invention will be explained. In this case, a so-called graduated PCR is employed, which is a detection means disclosed in a paper of Adleman (Science, 266, 1021-4).

In FIG. 32, the nucleic acid molecule showing a solution and generated by the computer has a sequence having 4 variables expressing T (true) or F (false) which are ligated from the 5′ end. The sequence of the solution is individually amplified by a PCR reaction using primers (shown in FIG. 32), which are capable of detecting all possible solutions. In the figure, a horizontal line drawn above a sequence denotes a complementary sequence. Assuming that the sequence (in the uppermost stage) representing a solution has been obtained, PCR products having the lengths shown in the figure can be obtained by a set of primers shown in the lowermost stage of FIG. 32. If the presence and absence of the products and the lengths thereof are checked by gel-electrophoresis, it is possible to know the sequence of a solution from the primer giving the PCR product.

The aforementioned functions are summarized in Table 3.

TABLE 3

get (T, +3), get (T, −s)
a DNA molecule containing (not containing) a
partial sequence s is picked up from a test
tube T
append (T, s, e)
Sequence s is added to the end of a DNA
molecule satisfying a terminal end-condition
e and present in a test tube T.
merge (T₁, T₂, Tₙ)
DNA molecules present in test tubes T₁ T₂ . . . Tₙ
are combined.
amplify (T₁ T₂, Tₙ)
DNA molecules present in a test tube T are
distributed into test tubes (T₁, T₂, Tₙ)
without changing the concentration.
detect (T)
DNA molecule present in a test tube T
is detected.

The main function of the program shown in Equation 4 is dna3sat. Function dna3sat includes the basic function described in the above and Function getuvsat formed on the basic function. First of all, the names of variables of the program will be explained.

A symbol T is an abbreviation of a tube. T₂ is a solution containing oligonucleotides expressing 4 of all possible false/true combinations formed of 2 variables x₁ and x₂. X₁^{T} is an oligonucleotide (of 22 nucleotides) representing that the value of x₁ is true. X₂^{F} is an oligonucleotide (22 nucleotides) representing that the value of x₂ is false. X₁^{T}X₂^{F}, one of oligonucleotides contained in the solution T₂, is a single-stranded oligonucleotide (44 bases) representing assignment x₁=1, x₂=0. Symbols j and k is are integers representing which stage of the loop the operation proceeds. More specifically, the symbol j represents which clause of the logical equation of a problem the operation is performed. The symbol k represents which of variables is calculated. The symbols u, v, w represent respective literals of each clause of the logical equation. In the case of Equation 2, the literals are u₁=x₁, v₂=x₂, w₁= [symbol] x₃. The line above sequence name X represents that the sequence is a complementary sequence. The T/F on the shoulder of sequence name X represents T or F, representing X^{T} or X^{F}.

Now, the program will be explained in order. In practice, an experimental design is made by the program interpreting portion. More specifically, the program interpreting portion develops a program function, conditional branch, and a “for” loop into a series of experimental operations. The logical equation of a problem is modified in advance to make processing easier by arranging the order of literals in the ascending order of subscripted letters of variables in each clause. In addition, a solution T₂ is prepared so as to contain 2 variables x₁ and x to which possible assignment is made.

In Function dna3sat, an initial “for” loop, k=3, that is, a loop for determining a value to be assigned to x₃, will be explained. First, T₂ is amplified by Function “amplify”. In this case, after amplification, T₂ is distributed into T_{w}^{T} and T_{w}^{F} having the same oligonucleotide. Three solution tubes are obtained in total. Subsequently, the operation goes to “for loop” of “j” within the “k” loop. Herein, sufficiency of the “j” th clause of the logical equation is evaluated. At the first conditional branch, provided that j=1, the third literal w₁ of the first clause is equal to x₃ is checked. To explain more specifically, the logical equation is previously checked in the electronic operation section to determine whether or not the operation of “then” onward is performed, thereby determining whether the operation of “then” onward is added to the experimental design. In the case where the third literal w₁ of the first clause is equal to [symbol] x₃ at the 2nd conditional branch within the loop, the operation of “then” onward is executed.

Function “getuvsat” is separately defined in the lower step, T_{w}^{T} tube containing an oligonucleotide (the content and concentration are the same as those of T₂) and produced by initial amplification is input to each of arguments, T. U₁(=x₁) is input to u, and v₁(=x₂) to v. A first Tᵤ^{T} solution is prepared by extracting an oligonucleotide containing X₁^{T} from the T_{w}^{T} solution. In this manner, the content of the solution Tᵤ^{T} is {x₁^{T}X₂^{T}, X₁^{T}X₂^{F}}. Second, a solution T′ᵤ^{F} is prepared by extracting an oligonucleotide not containing X₁^{T} from the solution T_{w}^{T}. In this manner, the content of T′ᵤ^{F} is {x₁^{F}X₂^{T}, X₁^{F}X₂^{F}}. Third, a solution Tᵤ^{F} is prepared by extracting an oligonucleotide containing X₁^{F} from the solution T′ᵤ^{F}. In this manner, the content of Tᵤ^{F} is {x₁^{F}X₂^{T}, X₁^{F}X₂^{F}}. In the program, there is a line reading “*can be omitted*”. However, it is apparent from the content of the solution prepared. Since this line is added so as not to generate an experimental error. The operation of this line is executed for precautionary purposes. When this line is omitted, T′_{U}^{F} must be substituted by T_{U}^{F}. Fourth, a solution Tᵥ^{T} is prepared by extracting an oligonucleotide containing X₂^{T} from the solution Tᵤ^{F}. In this manner, the content of Tᵤ^{T} is {x₁^{F}X₂^{T}}. Finally, Tᵤ^{T} and Tᵥ^{T} are mixed to prepare a T^{T} solution by Function “merge” and output by Function “return”. As a result, the T^{T} solution becomes {x₁^{T}X₂^{T}, X₁^{T}X₂^{F}, x₁^{T}X₂^{T}}. The T^{T} solution is renamed T_{w}^{T} and subjected to Function “dna3sat”. The content of the T^{T} solution is a set for assignment for x₁ and x₂. Since the first clause is x₁ [symbol] x₂ (x₁ and x₂ are first literal and second literal), even if the third literal [symbol] x₃ takes any value. From the above, Function “Getuvsat” screens an oligonucleotide showing assignment such that the clause exhibits true even if any value is assigned to the variable of the third literal of the clause where values have been assigned to two literals.

Next, in the k=3 loop of Function dna3sat, the value of j is incremented by one, that is, under the condition of j=2, the operation goes to the second clause. In this clause, since the third literal is [symbol] x₃ in the same as the in the first clause, the line which affects the conditional branch is “if-sentence” of the following step. Function “getuvsat” is executed in the same manner as in the first clause. In this case, care must be taken to the fact that T_{w}^{T} has been already produced in the previous step of j=1. In addition, v₂ is a literal including “negation”, that is, v₂= [symbol] x₂. Therefore, in the equation for obtaining Tᵥ^{T} of Function “getuvsat”, Function “get” is executed by substituting Xᵥ^{T} by X₂^{F}. In this case, the value of T_{w}^{T} newly obtained in the case of j=2 results in {x₁^{T}X₂^{T}, X₁^{T}X₂^{F}}.

In the same manner as mentioned above, the operation repeats until j=10. During the operation, for example, 5th clause onward, in the case where the third literal is k=4, the value of “j” is incremented, to thereby operate the next operation of the loop. After the operation of the loop corresponding to 10 clauses is completed, X₃^{T} expressing “true” is appended to the oligonucleotide of the 3′ end, X₂ of T_{w}^{T}. Furthermore, X₃^{F} expressing “false” is appended to the oligonucleotide of the 3′ end, X₂ of T_{w}^{F}. After these solutions obtained after the “append” operation, are merged and the operation goes to a loop of k=4. After completion of the k=4 loop, the operation exits from the loop. The solution of T₄ tube is processed by Function “detect”.

Since the calculation is performed as mentioned above, provided that the number of variables is n and the number of clauses is m , the execution number of each of basic commands is given by the following equation:

(n−2)×(amplify+2×append+merge)+m ×(3×get+merge)

This equation means that 3SAT problem is solved for time period substantially in proportional to the numbers of variables and clauses.

Furthermore, in the present invention, the detect portion is not always used for measuring the reaction results. The detect portion may be preferable if reaction results can be transmitted to the electronic operation section while they maintain their availability as they are or they may be changed or extracted into the forms which can be used by an operator. Furthermore, the detect portion may be preferable if it is in the measurable conditions. The measurement may be performed by an operator. Accordingly, in the present invention, the molecular operation section may provide products or results which can be used by an operator upon completion of the reaction. The product or results provided by the computer of the present invention can be most efficiently applied to diagnoses, treatments, drug designs, scientific studies, construction of biological data bases, and interpretation of biological information.

(4) Computation by the molecular computer

Examples of problems to be suitably solved by the molecular computer according to an aspect of the present invention include pure mathematic problems such as the NP complete problem and 3SAT, problems such as a genomic information analysis performed by inputting nucleic acid molecules, design for functional molecules and evaluation of a function, and problems which are difficult to be solved by an electronic computer.

Now, another example of genomic information analysis performed by the computer of the present invention will be described. First, genomic information is converted into a numerical DNA coding system assigned by orthonormal DNA nucleotide sequences. Thereafter, DNA computation is performed by using DCN codes in the same manner as in solving a pure mathematical problem. The genomic information obtained from the calculation results is analyzed.

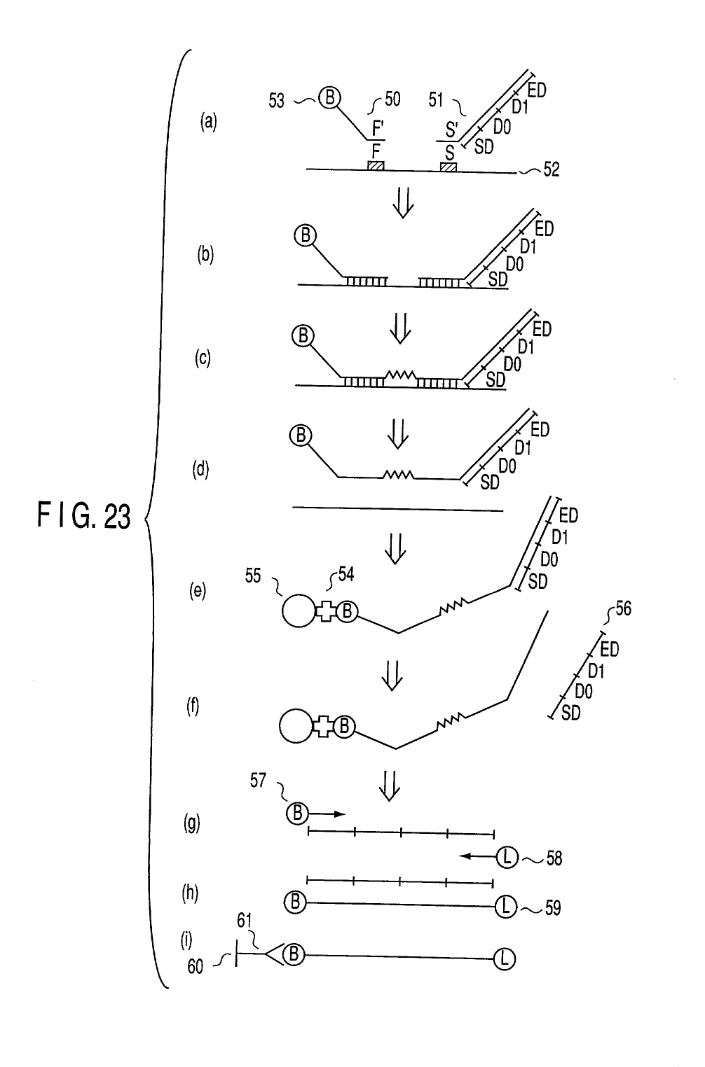

In this method, two types of nucleic acid probes as shown below, namely, probe A and probe B, will be used (FIG. 23a).

Probe A is formed of a sequence F′, which is complementary to a partial nucleotide sequence F of a target nucleic acid, and a binding molecule.

The binding molecule used herein is one of two substances having a high affinity with each other. Examples include biotin, avidin, streptoavidin and the like. The binding molecule may be directly bind to the sequence F′ or indirectly bind to the sequence F′ via an arbitrary sequence. As the arbitrary sequence used in the indirect binding, any nucleotide sequence having any number of nucleotides, may be used. Preferably non-complementary sequence to the nucleotide sequence of a target nucleic acid may be used.

Probe B is formed of a sequence S′, which is complementary to a partial nucleotide sequence S of the target nucleic acid, and a flag. The flag used in the embodiment is formed of a double strand. The double strand refers to an arbitrary sequence formed of a plurality of units. The Flag neither binds to the target nucleic acid nor exhibits any interaction thereto.

The sequences F′ and S′ to be used in the method of the present invention have at least one nucleotide, more preferably, at least 15 nucleotides.

The design of the units of a flag FL are shown in FIG. 31. Each of a plurality of units constituting a flag FL may contain at least 10 nucleotides, more preferably, about 15 nucleotides. The number of units of the flag FL is not limited but preferably 4 units in consideration of analysis. However the present invention is not limited to these condition.

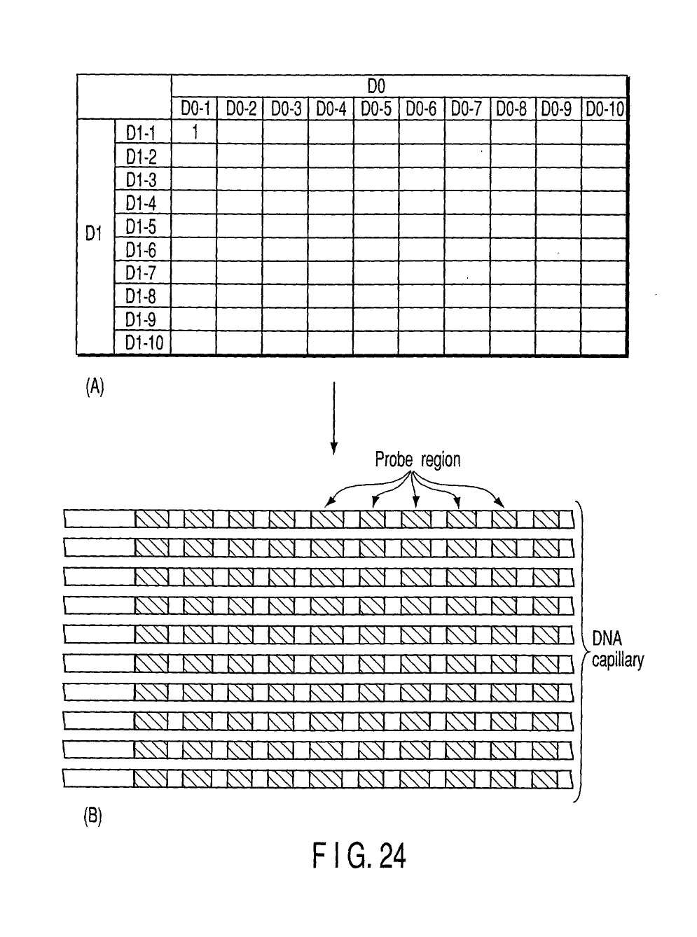

When a plurality of target nucleic acids are simultaneously detected, the flag FL is constructed by combining a plurality types of units. For example, the case where the flag FL of 4 units, SD, D0, D1 and ED, is designed, first, 22 types of units are designed. Of them, two types of units are selected. One is used as an SD unit serving as a primer. The other is used as an ED unit serving as another primer. Units D0, D1 are designed so as to differ depending upon the types of the target nucleic acids by selecting them from the remaining 20 types of units. As a result, 100 types of different nucleic acid sequences can be detected (FIG. 24A).

It is preferable to design 22 types of units by using orthonormal nucleotide sequences. The orthonormal nucleotide sequences have almost equal Tm values. Each of the orthonormal nucleotide sequences is also designed so as no stable hybrid can be formed with a sequence except for the complementary sequence. In addition, no stable secondary structure is formed, so that a hybrid formation with the complementary sequence will not be inhibited. In this way, the rate of mishybridization can be reduced at the time of final detection. As a result, the detection accuracy can be improved and the detection time can be shortened. If the number and types of units are increased, it is possible to detect different nucleic acid sequences of 10000 types.

Referring now to FIG. 23, the method of the present invention will be further specifically explained. FIG. 23a shows a flag FL consisting of 4 units. The 4 units include an SD unit, D0 unit, S1 unit and ED unit. The SD unit and ED unit serve as primers in a polymerase chain reaction (hereinafter, referred to as “PCR amplification” or “PCR”), The D0 unit and D1 unit serve as recognition portion for recognizing a type of target nucleic acid. Each of the units is used as a reading frame in a later step.

Detection is performed by blending the probe A and probe B with a target nucleic acid (FIG. 2, 3a). The target nucleic acid contained in a sample used herein may contain a plurality of target nucleic acid molecules. In this case, if the types of the target nucleic acids to be detected are 100 types or less, D0 unit is selected from 10 types of D0-1 to D0-10 units and D1 unit is selected from 10 types of D1-1 to D1-10 units (FIG. 24A).

Subsequently, probe A and probe B are incubated for a predetermined time under the conditions suitable for hybridization to thereby perform hybridization (FIG. 23b). The hybridization conditions are as shown in FIG. 1.

By the hybridization reaction thus performed, both probe A and probe B bind to the same target nucleic acid (FIG. 23b).

Subsequently, probe A and probe B hybridized to the target nucleic acid are ligated (FIG. 23c). The ligation conditions are as shown in Example 1.

The Tm value of a flag FL is set at a temperature higher than those of sequence F′ and S′. This is made to prevent denaturation of the flag (which deteriorates sensitivity) in heating or cooling step at the time of hybridization, ligation and denaturation reaction.

Then, the obtained information of the flag FL is subjected to B/F separation. To be more specific, the binding molecule of a probe (A+B) is bonded to a solid carrier via another binding molecule paired up with the binding molecule (FIG. 23e).

As the solid carrier, a substrate, particles such as beads, container, fiber, tube, filter, affinity column, and electrode may be used. Of them, beads are preferably used.

Then, the flag FL of probe (A+B) captured by the binding molecule is denatured as it is to obtain a single strand (FIG. 23f). The obtained single-stranded sequence FL′ contained in a liquid phase is subjected to PCR amplification (FIG. 23g). Since two primer sequences SD and ED are arranged in the flag FL, as mentioned above, the PCR amplification can be readily performed by use of the primer sequences. In this case, it is preferable that biotin be bounded to one of the two primers, for example, the SD sequence. The detained PCR conditions vary depending upon a designed FL.

After completion of the PCR reaction, the double stranded PCR product is recovered by binding it to the binding molecule fixed on the solid carrier (FIG. 23h). The solid carrier is a substance which can pair up with the binding molecule. Further, the sequence FL′ is removed by denaturation. As a result, a single-stranded sequence FL alone is recovered on the solid carrier (FIG. 23i).

Subsequently, the single-stranded flag sequence FL on the carrier is analyzed. First, the solid carrier having a single stranded flag sequence FL bound thereto is divided into 10 fractions (D1 unit consists of D1-1 to D1-10). One of sequences D1-1′ to D1-10′ tagged with a marker molecule and all D0′ sequences (D0-1′ to D0-10′) are added to allow D0′ sequences and the selected D1 sequence to hybridize to the flag sequence FL.

Subsequently, the two nucleic acid molecules hybridized are ligated. In this case, the ligation conditions and the marker substances are as defined above. Thereafter, the ligated molecule is recovered in a liquid phase by denaturation.

The obtained labeled nucleic acid molecule is analyzed by hybridizing it to a DNA chip or a DNA capillary in which nucleic acid molecules D0-1 to D0-10 have been fixed on a solid phase. Particularly, the DNA capillary is useful, since 10 reactions of D0-1 to D0-10 can be treated at the same time. It the DNA capillary is used, the analysis can be performed more easily.

As an example, the case of the flag FL which is designed by using 10 types of sequences, D0-1 to D0-10, and 10 types of sequences, D1-0 to D1-10 will now be explained. Since the sequence D0-1 is immobilized at position 1 of FIG. 24A, the nucleic acid molecule 63 ligated to a D1-1′ molecule labeled with a marker molecule, is hybridized with the sequence D0-1 at position 1. Similarly, since a D0 sequence is immobilized to a corresponding position n of the column, the nucleic acid molecule ligated to the D1′ molecule (i.e., a molecule corresponding to a row) is hybridized with the position n of the column. If such a matrix arrangement is applied to the DNA capillary described later, the analysis can be readily performed (FIG. 24B).

In this example, 10 types of units are used. The types of units are not limited to 10. Less than 10 and more than 10 may be used.

The “DNA capillary” used herein is a device for detecting a target nucleic acid. In the DNA capillary, a complementary sequence to the target nucleic acid is fixed. In this device, the target molecule may be detected by binding it to the complementary sequence.

As shown in FIG. 24B, if a plurality of DNA capillaries having different probes (arranged at hatched portions) are simultaneously used, a plurality of target nucleic acids can be detected at the same time.

In the method of this example, since an orthonormal nucleotide sequence is used as each unit of the flag sequence FL, hybridization can be uniformly performed under the same conditions, e.g., a reaction temperature. Therefore, the detection can be made with a high accuracy while preventing mis-hybridization. Furthermore, since numerous analyses can be performed simultaneously under the same conditions, the time required for the detection can be reduced.

According to the method of the present invention, complicated genomic information expressed by a nucleotide sequence of DNA can be converted into numerical values. Moreover, if calculation is made by using a DNA molecular reaction, analysis of various types of information and complicated genetic information mutually associated can be easily made. In addition, after a nucleic acid is encoded, the nucleic acid can be amplified based on the code. Therefore, even if the target sequences are present in low copy numbers, the target sequences can be accurately and quantitatively detected. Furthermore, it is possible to compress numerous data by encoding the sequence. Therefore, detection can be made by using a fewer number of devices, such as a DNA chip or a capillary array.

The “encode reaction” used herein refers to converting a nucleotide sequence to a code represented by orthonormal nucleic sequences. The encode reaction is performed in the steps of FIG. 23a to f.

The “decode reaction” used herein refers to reading the code which has been converted as mentioned above, thereby reproducing the original information.

In this method, it is possible not only to detect one type of target nucleic acid as mentioned above and but also detect a plurality of types of target nucleic acids by performing the same process using a plurality of types flag sequence designed.

### EXAMPLES

1. Molecular computation

The program of Equation 4 explained in the above was implemented by a molecular computer of the present invention. The computer implementing the program was prepared by modifying an automatic nucleic acid extraction device SX8G manufactured by PSS Co., Ltd. (Precision System Science). The device of the present invention includes a computer (electronic operation section) using windows 98 as an operation system equipped with Pentium III CPU (manufactured by Intel) as a control unit, a reagent vessel and reaction vessel used in an actual reaction, pipette for control the XYZ position, spare pipette tip, an experimentation robot (i.e., molecular operation section) comprised of a thermal cycler (PYC-200 manufactured by MJ Research) whose temperature can be controlled by a computer. FIG. 28 shows a top view of arrangement of reaction members in the molecular operation portion.

A solution was transferred between the reaction vessels. An oligonucleotide was extracted with streptoavidin beads by using a specific tip of the pipette and a permanent magnet which moved closer to or moved away from the tip. However, two operations were not performed within the aforementioned system. One is adding a small amount of enzyme (e.g., about 1 μL), which was manually performed. The other is capillary-gel electrophoresis by Function “detect”, which was performed by a P/ACE-5510 capillary electrophoresis system (manufactured by Beckman·Corlter).

Reagents and the initial arrangement of the reagents will be described. Since ligase enzyme was dispensed by hand as described above, it was stored outside the apparatus. The enzyme was added by a pipette “pipetto-man” 2 μL(manufactured by Gilson) was used.

Reference numeral X indicates the length of the oligonucleotide indicating a variable. In this case, the oligonucleotide is formed of 22 nucleotides. The “X₁^{T}X₂^{F}” means an oligonucleotide in which X₁ and X₂ are arranged in this order from the 5′ end. X₁^{T} herein is an oligonucleotide indicating that the sequence X₁ is “true”. X₂^{F} herein is an oligonucleotide indicating that the sequence X₂ is “false”. If the name of a sequence is surrounded by parentheses [ ], the sequence is a complementary sequence to the original sequence. More specifically, [X₁^{F}x₂^{F}] denotes a sequence complementary to X₁^{F}x₂^{F}. Actually, the sequence consists of a sequence complementary to X₂F and a sequence complementary to X₁^{F} which are arranged in this order from the 5′ end.

A T₂ solution (5 pmol of each of X₁^{T}x₂^{T}, X₁^{F}x₂^{T}, X₁^{T}x₂^{F}, X₁^{F}x₂^{F} was dissolved in 20 μL of a ligation buffer solution) was stored in MTP 35 (multi-titer plate 35). A buffer solution for a ligation reaction, streptoavidin magnetic beads, a B & W solution was stored in MTP 37 shown in FIG. 28. The B & W solution contained 1 M NaCl in TE solution. The B & W solution was used by capturing a biotin-labeled oligonucleotide by streptoavidin magnetic beads and used in a hybridization reaction.

In MTP 35, placed were biotinylated oligonucleotides (whose 5′ end was labeled with biotin), append oligonucleotides (whose 5′ end was phosphorylated) and biotinylated ligation oligonucleotides (whose 5′ end was labeled with biotin). The biotinylated oligonucleotides [bX₁^{T}], [bX₂^{T}], [bX₃^{T}], [bX₄^{T}], [bX₁^{F}], [bX₂^{F}], [bX₃^{F}], [bX₄^{F}] were prepared by dissolving 10 pmoL of each of the biotinylated oligonucleotides in 20 μL of B & W solution. The append oligonucleotides pX₃, pX₃^{T}, PX₃^{F}, pX₄^{F}, pX₄^{T}, were prepared by dissolving 10 pmoL of each of the append oligonucleotides in 20 μL of ligation buffer solution. The biotinylated ligation oligonucleotide [bX₂^{T}X₃^{T}], [bX₂^{F}X₃^{T}], [bX₂^{T}X₃^{F}], [bX₂^{F}X₃^{F}], [bX₃^{T}X₄^{T}], [bX₃^{F}X₄^{T}], [bX₃^{T}X₄^{F}], [bX₃^{F}X₄^{F}] were prepared by dissolving 10 pmol of each of the biotinylated ligation oligonucleotides in 20 μL of ligation buffer solution. These oligonucleotide solutions and buffer solutions were stored at room temperature during the operation of the molecular operation section. Ligase, PCR polymerase for use in Function “detect” were stored in ice outside the device. The buffer solution for PCR reaction is stored at room temperature.

The operation of the device in the reaction corresponding to each Function will be explained in order.

(a) Function “amplify”

Each oligonucleotide was amplified by PCR by using a solution of an argument at the left end, as a template. The concentration of the oligonucleotide thus amplified was set at the same as that of an original solution and divided into a plurality of solutions. In the strict sense, the oligonucleotide should be amplified after the oligonucleotide concentration of the original solution was measured. Actually, 5 pmol of each oligonucleotide was dissolved in 20 μL of the T₂ solution in the initial step. Therefore, the concentration of each solution was set so as to contain several pmol of each oligonucleotide in 20 μL of the solution. More specifically, assuming that the concentration of an oligonucleotide per solution is 2 pmol, the composition of a PCR reaction solution is as follows:

| | |
|---|---|
| Polymerase enzyme | 0.5 μL (2.5U) |
| (Takara Shuzo Co., Ltd.) | |
| Solution for DNA | containing about 1 fmol |
| amplification | of each oligonucleotide |
| dNTP solution mixture | 8 μL (2.5 mM, appendix) |
| Reaction buffer | 10 μL (10 X dilution, |
| | appendix) |
| Primer | 5 pmol of each of |
| | forward and reverse |
| | primers per |
| | oligonucleotide |
| Sterilized distilled | Added up to a total |
| water | amount of 100 μL |

Amplification was performed by using a Pyrobest™ DNA polymerase PCR amplification kit (Takara Shuzo Co., Ltd.). The reaction temperature conditions were as follows:

1. 95° C. for 30 seconds

2. 50° C. for 30 seconds

3. 72° C. for 60 seconds

A cycle of 1-3 steps was repeated for 30 times.

The amount of PCR solution was varied depending upon the number of solutions to be divided. After PCR, a PCR product was captured by streptoavidin magnetic beads (Roche diagnostics) from the PCR reaction solution. 50 μL (0.5 mg) of magnetic bead original solution was taken and magnetic beads only were fixed by a magnet from the magnetic bead original solution. The magnetic bead original solution was replaced with 50 μL of the B & W solution. The magnetic bead solution thus prepared was mixed with 50 μL of the PCR reaction solution to capture a PCR product. While the PCR product was captured, the solution was replaced with 50 μL of the B & W solution and heated to 88° C. to dissociate a single-stranded oligonucleotide to get it. The operation mentioned above was not performed by an experimental apparatus but manually performed.

(b) Function “get”

Get (T, +S) and get (T, −S) were simultaneously performed in a series of operations.

(1) 50 μL of extraction solution T was prepared and supplied to thermal cycler H.

(2) 50 μL of the B & W solution containing 20 pmol of biotinylated oligonucleotide which was complementary to a target sequence was further added to the solution T. In this manner, a first hybridization reaction was performed (in accordance with the following reaction conditions, 1 and 2). An aliquot was taken by a pipette from each of MTPs and a reaction was allowed to proceed under the reaction temperature conditions:

1. 95° C. for one minute

2. 25° C. for 10 minutes (the temperature is decreased at a rate of 10° C./minute from step 1 to step 2)

3. 56° C. for 3 minutes (the temperature is increased at a rate of 10° C./minute from the step 2 to step 3)

4. 75° C. for 3 minutes (the temperature is increased at a rate of 10° C./minute from step 3 to step 4)

In accordance with the aforementioned menu, the thermal cycler was controlled while providing a sufficient time for pipetting.

(3) In the middle of step 2 of decreasing the temperature, 50 μL of the B & W solution in which 50 μL of the magnetic bead original solution was dispersed, was mixed to capture a hybrid of the biotinylated oligonucleotide on the magnetic beads. The magnetic beads were again returned to 96-hole MTP38 of the thermal cycler.

The magnetic bead solution was once suctioned by the pipette of the apparatus and then stored in a space of the tip of the pipette. At the time, a movable permanent magnet attached to the pipette was approached to the tip storing the magnetic bead solution to collect beads. While colleting the beads, the solution was discharged from the pipette and a fresh solution was sucked. In this way, the solution was substituted with the fresh solution and the double stranded nucleic acid was dissociated into complementary strands. To dissolve the magnetic beads into the solution, pipetting was performed for a few times while staying the permanent magnet away from the tip. In this operation, the magnetic beads were sufficiently stirred and dispersed in the magnetic bead solution.

(4) The operation goes to the temperature conditions 3. In the step 3, cold wash was performed to collect an oligonucleotide unhybridized. The oligonucleotide was extracted into 50 μL of the B & W solution at 56° C. and output into the MTP of E. In this way, an output oligonucleotide solution corresponding to get (T, −S) was obtained.

(5) In the hot-wash step of the temperature condition 4, the temperature was further increased. The oligonucleotide captured by the magnetic beads was extracted into 50 μL of the B & W solution in the same manner as in the cold wash, and then output into MTP 35. In this way, an output oligonucleotide solution of get (T, +S) was obtained.

(c) Function “merge”

This Function was executed by an extremely simple pipetting operation. More specifically, the solutions to be mixed were sucked by a pipette and collectively placed in a single well of an MTP and mixed them.

(d) Function “Append”

The reaction is shown in FIG. 30. The “Append” reactions of different oligonucleotides were simultaneously performed in different reaction tubes.

(1) 20 μL of the solution to be subjected to the append reaction was sucked by a pipette from the MTP 35 and supplied into the thermal cycler 38. In addition, the following solutions required for the “append” reaction was transferred to the reaction well 38.

In the append reaction, Taq ligase and a specific buffer solution (New England Bio Labs) were used.

The reaction solution is as follows:

| | |
|---|---|
| Taq ligase (NEB) | 0.5 μL (20 U) |
| (Takara Shuzo Co., Ltd.) | |
| Reaction solution of DNA | original solution: 20 μL |
| Ligation buffer | 12 μL |
| | (10 X dilution is used, |
| | appendix) |
| ligation oligonucleotide | 10 pmol for each |
| “Append” oligonucleotide | 10 pmol for each |
| Sterilized distilled | Added up to a total |
| water | amount of 120 μL |

(2) A ligation reaction was performed. The thermal cycler was controlled as follows. The ligation reaction was performed under the temperature conditions 1 and 2. The reaction temperature conditions are as follows:

1. 95° C. for one minute

2. 58° C. for 15 minutes (the temperature is decreased at a rate of 10° C./minute from step 1 to step 2)

3. 25° C. for 10 minutes (the temperature is decreased at a rate of 10° C./minute from the step 2 to step 3)

4. 70° C. for 3 minutes (the temperature is increased at a rate of 10° C./minute from step 3 to step 4)

5. 74° C. for 3 minutes (the temperature is increased at a rate of 10° C./minute from step 4 to step 5)

6. 88° C. for 3 minutes (the temperature is increased at a rate of 10° C./minute from step 5 to step 6).

(3) After the temperature was decreased to 25° C. in the reaction temperature condition 3, the capturing operation was performed by the magnetic beads. At this time, the solution dispersing the magnetic beads was discarded, a ligation buffer solution was directly sucked by a pipette from the thermal cycler 38 while the beads were stored in a tip. More specifically, the capturing reaction was performed in the ligation buffer solution. Since the buffer solution is not a specific solution for capturing reaction, sucking and discharging were performed for 60 times as a precaution, to obtain a sufficient performance of capturing. The obtained magnetic beads were stored in the thermal cycler 38.

(4) Subsequently, first cold wash was performed. Based on the length of the nucleic acid hybrid and the salt concentration of the solution, the cold wash was preferably performed at 70° C. When the temperature increased to 70° C. in step 4, the magnetic bead solution was sucked from the thermal cycler 38. The magnetic beads was collected by a permanent magnet and the solution alone was discharged into the MTP 35. The solution was a waste.

(5) 50 μL of the B & W solution of the MTP 37 was sucked by a pipette and the magnetic beads was dispersed in the B & W solution by moving away the permanent magnet. The resultant solution was returned into the thermal cycler 38. In the thermal cycler 38, second time cold wash was performed at reaction temperature 5. In the same manner as in the step (4), when the temperature reached an ideal value, the solution was sucked from the thermal cycler 38 and the beads were collected by a permanent magnet. Thereafter, only the B & W solution was discharged into the MTP 35. The B & W solution was a waste.

(6) Thereafter, 50 μL of the B & W solution in the MTP 37 was sucked again by a pipette. The magnetic beads were dispersed in the B & W solution by leaving away the permanent magnet and returned into the thermal cycler 38. When the B & W solution reached the reaction temperature 6 in the thermal cycler 38, a single-stranded oligonucleotide was dissociated from the “append” reaction previously performed. The dissociated single-stranded oligonucleotide was sucked from the thermal cycler 38 and the beads were collected by the permanent magnet. Only the solution was discharged into MTP 35 and stored therein.

(e) Function “detect”

Function “detect” was executed by the reaction shown in FIG. 32 and the detection by capillary gel electrophoresis. Graduated PCR was performed by using the finally obtained solution which might contain an oligonucleotide exhibiting a solution, as a template. PCR was performed by dividing the reaction solution depending upon primer sets. The sequence of the solution was checked by detecting the presence/absence and the length of a PCR product for each primer set.

(1) The oligonucleotide solution which might contain a solution, was used as a template. Since the concentration of oligonucleotide was maintained by “amplify” in the program, the concentration of the template was estimated from the concentration of oligonucleotide. Based on the estimation, the amount of the solution was determined.

The composition of the PCR solution is:

| | |
|---|---|
| Polymerase enzyme | 0.5 μL (2.5 U) |
| (Takara Shuzo Co., Ltd.) | |
| DNA Solution | containing about 1 fmol |
| to be amplified | of each oligonucleotide |
| dNTP solution mixture | 8 μL (2.5 mM, appendix) |
| Reaction buffer | 10 μL |
| | (10 X dilution, |
| | appendix) |
| Primer | 5 pmol of each of |
| | forward and reverse |
| | primers per |
| | oligonucleotide |
| Sterilized distilled | Added up to a total |
| water | amount of 100 μL |

Amplification was performed by a Pyrobest DNA polymerase PCR amplification kit ((Takara Shuzo Co., Ltd.).

The reaction temperature conditions were as follows:

1. 95° C. for 30 seconds

2. 50° C. for 30 seconds

3. 72° C. for 60 seconds

A cycle of 1-3 steps were repeated for 30 times.

The primers used in this experiment are the following 12 sets:

(X₁^{T}, [X₂^{T}]), (X₁^{T}, [X₂^{F}]), (X₁^{T}, [X₃^{T}]), (X₁^{T}, [X₃^{F}]), (X₁^{T}, [X₄^{T}]), (X₁^{T}, [X₄^{F}]), (X₁^{F}, [X₂^{T}]), (X₁^{F}, [X₂^{F}]), (X₁^{T}, [X₃^{T}]), (X₁^{F}, [X₃^{F}]), (X₁^{F}, [X₄^{T}]), (X₁^{F}, [X₄^{F}]).

These were stored in different wells of the MTP 35. At the PCR reaction, the primer solution was sucked by a pipette and discharged into different wells of the thermal cycler 38. After the solution required for the PCR reaction was added, the thermal cycler 38 was operated as designed. In this manner, the reaction was completed. This operation can be easily performed automatically. The loading of a sample into a capillary in the capillary gel electrophoresis device can be performed automatically. In the capillary gel electrophoresis, ds1000 gel kit (manufactured by Beckmann/Coalter) was used.

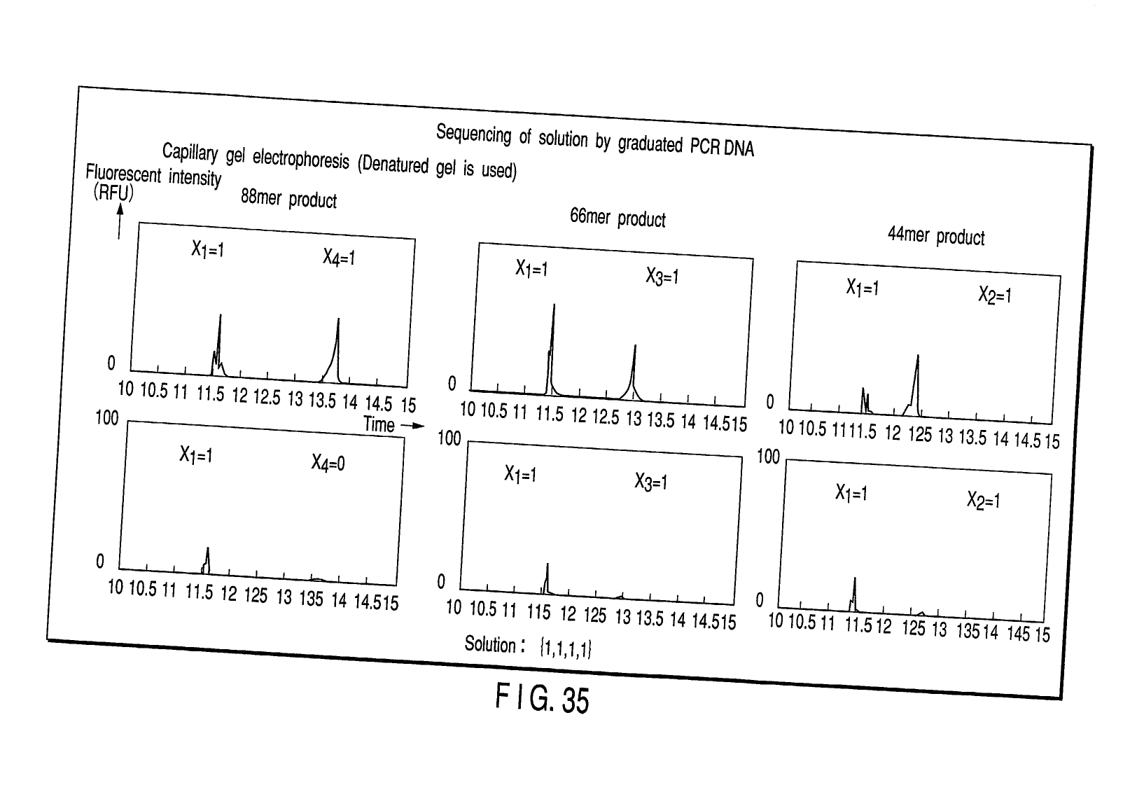

Since the primer X₁^{T} was labeled with FITC, an electrophoresis image was observed. In the “detect” operation mentioned above of this embodiment, the capillary electrophoresis was not performed automatically but manually performed. The results obtained by executing individual functions of the program in the method mentioned above, are shown in FIG. 35.

To demonstrate the efficiency of operation paradigm such as genomic information analysis by a molecular computer, an experiment was performed by analyzing the gene expression by using DNA computer. The calculation was performed by using basic commands “get”, “append”, “amplify”, “merge” and “detect”, which were usually used when 3SAT problem was solved by dynamic programming. The first operation reaction was an encode reaction, which was performed in a single test tube. The information of a transcriptional product of a gene was converted into DCN by “append” command. The conversion table was expressed by an adapter molecule Ai. More specifically, the conversion table is expressed one-by-one combination of 2 sets of DCNs. The present inventors have already obtained 200 types of DCNs in this way. Therefore, if two-set centesimal is used in combination, 10,000 types of genes can be encoded by DCNS. Thereafter, amplification is performed by “amplify” command and the amplified product is distributed into n test tubes. Finally, the n test tubes were subjected to a decode reaction of DCN by “append” and “get” commands. The decode reactions of the n test tubes were performed simultaneously. When an experiment was performed by using a transcriptional product of transplanted fragment-to-host disease as cDNA input data, it was demonstrated that the calculation reaction was performed specifically and quantitatively.

The method of analyzing gene expression by operating DCN by the DNA computer has several advantages compared to the method of directly analyzing a transcriptional molecule by a DNA chip. First, since gene information is converted into DCNs having the uniform nature, it is possible to analyze the DCN after amplifying it without changing an original frequency of gene expression. As the DNA chip for performing “get” command simultaneously in parallel in the DCN decode reaction, the same DNA chip may be used as long as calculation is performed by using the same DCN code-conversion system. Second, the number of DNA probes can be significantly reduced. Therefore, labor and cost for preparing a DNA chip can be greatly reduced. Furthermore, the hybridization reaction of an orthonormal probe is optimized, so that calculation can be performed accurately.

According to the aspect of the present invention, the computer of the present invention takes advantages of not only the molecular computer which achieves high parallel computation, but also the electronic computer which complements the functions not be attained by the molecular computer. It is therefore not necessary for an operator to make an experimental design and to perform assignment of coding molecules for molecular computation.

Furthermore, if the gene analysis was performed by the computer of the present invention, it is possible to evaluate the presence or absence of a target nucleic acid having a specific sequence, and further determine the geno type and the expression state of a gene, based on the presence/absence evaluation, simply and at a low cost, while minimizing experimental errors.

Furthermore, the present invention provides the following method and a molecular computation software based on the above. More specifically, the present invention provides a molecular computation method characterized in that the electronic operation section and the molecular operation section are integrally operated based on the molecular information expressed in the form which is capable of being recognized by an electronic program.

The present invention further provides a software applicable to the molecular computer including the electronic operation section and the molecular operation section. The molecular computation software has a feature in that it can be applied to the electronic operation section and/or the molecular operation section, and that calculation operations of the electronic operation section and the molecular operation section can be performed by using data-form electrically recognizable by each operation section. To be specific, the present invention provides a molecular computation software having a function for converting the data obtained by calculation performed at the molecular operation section into data form which is applicable to an electronic program of the electronic operation section. To be more specific, the present invention provides a molecular computation software having a function for converting the data, which is obtained by calculation operation performed in the electronic operation section, into a data-form applicable to computation operation of the molecular operation section.

The computer of the present invention can be easily operated by using the molecular operation software of the present invention. The molecular operation software can be used if the computer of the present invention is integrally controlled, if a part of the structural element is independently controlled, or if some parts of the structural elements are controlled in combination.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of processing information by using an operational nucleic acid, comprising
(a) converting arbitrary information into a nucleic acid molecule;
(b) hybridizing the nucleic acid molecule obtained in said (a) to an operational nucleic acid designed so as to express a logical equation indicating a condition to be detected, and extending the nucleic acid molecule hybridized; and
(c) detecting a binding profile of the nucleic acid molecule included in the nucleic acid molecule extended in said (b), thereby evaluating whether a solution of the logical equation is true or false.

2. The method of processing information according to claim 1, wherein said nucleic acid molecule is an orthonormal nucleic acid.

3. The method of processing information according to claim 1, wherein said operational nucleic acid is configured of a plurality of sequence units, a sequence of each of the sequence units and the arrangement of the sequence units can be designed in accordance with the logical equation, and true or false of the logical equation is evaluated based on the binding of the nucleic acid molecule to each unit and extension thereof.

4. The method of processing information according to claim 1, wherein said operational nucleic acid is configured of a plurality of sequence units and a marker binding portion, the sequence of each of the sequence units and the arrangement of the sequence units can be designed in accordance with the logical equation, when said nucleic acid molecule binds to one of the units and is extended so that a marker does not bind to the marker binding portion, true or false determination of the logical equation is made whether or not a marker binds to the marker binding portion.

5. The method of processing information according to claim 1, wherein said operational nucleic acid is configured of a plurality of sequence units and a marker binding portion, a sequence of each of the sequence units and the arrangement of the sequence units can be designed in accordance with the logical equation which is formulated on the basis of the combination of presence and absence of a target nucleic acid, when said nucleic acid molecule binds to one of the units and is extended so that a marker does not bind the marker binding portion, true or false determination of the logical equation is made whether or not a marker binds to the marker binding portion.

6. A method of processing information using an operational nucleic acid, comprising
(a) selecting target sequences, and further selecting “presence” or “absence” of the target sequences as a condition, formulating a logical equation based on the selected sequences and combination of the presence or absence of the target sequences selected, and designing and preparing an operational nucleic acid in accordance with the logical equation;
(b) when the target sequence selected in said (a), is present, information “the target sequence is present” being converted into a “presence molecule”, whereas, when the target sequence is absent, information “the target sequence is absent” being converted into an absence molecule;
(c) hybridizing a presence/absence oligonucleotide previously prepared on the basis of the condition selected in said (a) with the presence molecule obtained in said (b) and extending the presence molecule;
(d) after step (c), recovering a single-stranded presence/absence oligonucleotide which has failed in forming a double strand because a desired information is absent;
(e) hybridizing the absence molecule to the presence/absence oligonucleotide recovered in (d), thereby extract the absence molecule;
(f) hybridizing the presence molecule and absence molecule extracted in said (b) and (e), respectively, to the operational nucleic acid prepared in said (a) and expending the presence molecule and absence molecule; and
(g) detecting binding profiles of the presence molecule and the absence molecule in the extended molecule obtained in said (f), thereby evaluating true or false of a solution of the logical equation.

7. The method of processing information according to claim 4, wherein said presence molecule and said absence molecule are orthonormal nucleic acids.

8. The method of processing information according to claim 4, wherein said operational nucleic acid is configured of a plurality of sequence units, a sequence of each of the sequence units and the arrangement of the sequence units can be designed in accordance with the logical equation, and true of false of the logical equation is evaluated based on the binding of the nucleic acid molecule to each unit and extension thereof.

9. The method of processing information according to claim 4, wherein said operational nucleic acid is configured of a plurality of sequence units and a marker binding portion, a sequence of each of the sequence units and the arrangement of the sequence units can be designed in accordance with the logical equation, when said nucleic acid molecule binds to one of the units and is extended so that a marker does not bind to the marker binding portion, true or false determination of the logical equation is made whether or not a marker binds to the marker binding portion.

10. The method of processing information according to claim 4, wherein said operational nucleic acid is configured of a plurality of sequence units and a marker binding portion, a sequence of each of the sequence units and the arrangement of the sequence units can be designed in accordance with the logical equation which is formulated on the basis of the combination of presence and absence of a target nucleic acid, when said nucleic acid molecule binds to one of the units and is extended so that a marker does not bind to the marker binding portion, true or false determination of the logical equation is made whether or not a marker binds to the marker binding portion.

11. The method of processing information according to claim 4, wherein said molecule is a nucleic acid.

12. A method of processing information using an operational nucleic acid for evaluating a logical OR or logical AND which corresponds to the presence or absence of the nucleic acid having a specific sequence or using an operational nucleic acid for evaluating a logical OR and a logical And.

13. A molecular computer having an electronic operation section and molecular operation section, wherein said electronic operation section controls a function of molecular operation section substantially, and the molecular operation is performed under control of the electronic operation section.

14. A molecular computer comprising an electronic operation section and a molecular operation section;
wherein, in said electronic operation section, a constant and a variable of a computation program are converted into coding molecules, and a procedure and a function of the computation program are converted into an operation reaction; implementation steps of the operation reaction are prepared from the computation program; and
in said molecular operation section, in which coding molecules are stored, the operation reaction of the coding molecules is performed in accordance with the implementation steps, whereby the results of the operation reaction are obtained or further detected.

15. A molecular computer comprising an electronic operation section and a molecular operation section;
wherein said electronic operation section comprises
means inputting a computation program, a constant and a variable of a computation program;
means converting the constant and variable of the computation program into a coding molecule;
means converting a procedure and a function of the computation program into a corresponding operational reaction of a coding molecule;
performing an operation of a part of the computation program;
means preparing an implementation procedure of the operation reaction in accordance with the computation program or the operation results of the molecular operation section; and
means controlling the operation reaction to be performed in the molecular operation section in accordance with the implementation procedure of the operation reaction; and
said molecular operation section comprises
operation means performing the operation reaction by using the coding molecule; and
detection means detecting the operation result performed by the operation means.

16. A molecular computer comprising an electronic operation section and a molecular operation section;
wherein said electronic operation section comprises
means inputting a computation program, a constant and a variable of a computation program;
means converting the constant and variable of the computation program into a coding molecule;
means converting a procedure and a function of the computation program into a corresponding operational reaction of a coding molecule;
performing an operation of a part of the computation program;
means preparing an implementation procedure of the operation reaction in accordance with the computation program or the operation results of the molecular operation section; and
means controlling the operation reaction to be performed in the molecular operation section in accordance with the implementation procedure of the operation reaction; and
means displaying the operation results detected by the molecular operation section; and
said molecular operation section comprises
operation means performing the operation reaction by using the coding molecule; and
detection means detecting the operation result performed by the operation means.

17. A molecular computer comprising
input means inputting a computation program for computation;
storage means storing the computation program input
operation means operating a part of the computation program;
storage means storing a molecule conversion table for assigning the computation program, and a constant and a variable of the computation program to coding molecules;
conversion means converting the computation program, and the constant and the variable of the computation program to coding molecules by reading the molecule conversion table and screening and reading out corresponding data stored therein;
synthesis means synthesizing the coding molecule;
storage means storing a procedure conversion table for converting the computation program into an experimental operation of the coding molecule;
plan preparation means preparing an experimental design by reading out the procedure conversion table, screening and reading out the corresponding data and converting the corresponding data into an experimental operation;
automatic control means outputting a driving signal in accordance with the experimental means prepared;
experimental means operating the experimental operation in accordance with the driving signal from the automatic control means by using the coding molecule synthesized;
detection means detecting the coding molecule obtained from the experimental operation;
processing means processing detection results into a form written in the computation program; and
an output means outputting results obtained by the processing means.

18. The molecular computer according to any one of claims 13 to 16, wherein said molecular operation section has a means for synthesizing the coding molecule.

19. The molecular computer according to any one of claims 13 to 17, wherein said molecule is a nucleic acid.

20. A molecular computation method integrally functioning an electronic operation section and a molecular operation section on the basis of molecular information recognizable by an electric program.

21. A molecular computation method integrally functioning an electronic operation section and a molecular operation section on the basis of molecular information recognizable by an electric program.

22. A software applicable to a molecular computer comprising an electronic operation section and a molecular operation section, wherein said software is applied to the electronic operation section and/or the molecular operation section; and an operation to be performed in the electronic operation section and an operation to be performed in the molecular operation section are allowed to function in an operation section of each of the electronic operation section and the molecular operation section, in the form of data electrically recognizable.

23. The software applicable to a molecular computer according to claim 22, comprising a function for converting data obtained at the molecular operation section into data form applicable to an electric program of the molecular operation section.

24. The software applicable to a molecular computer according to claim 22, comprising a function for converting data obtained at the electronic operation section into data form applicable to an operation of the molecular operation section.
